(19) United States
(12) Patent Application Publication
(10) Pub. No.: US 2025/0076292 A1
(43) Pub. Date: Mar. 6, 2025

(54) SYSTEM FOR THE SIMULTANEOUS DETECTION OF MULTIPLE ANALYTES

(71) Applicant: Foss Analytical A/S, Hilleroed (DK)

(72) Inventors: Tamir GIL, Hilleroed (DK); Steen Hauge MATTHIESEN, Hilleroed (DK); Erika Claudia LANTOS, Pecs (HU); Gyorgy NAGYERI, Pecs (HU)

(73) Assignee: Foss Analytical A/S, Hilleroed (DK)

(21) Appl. No.: 18/561,163

(22) PCT Filed: Jul. 12, 2022
(86) PCT No.: PCT/IB2022/056420

§ 371 Date: Nov. 15, 2023

(30) Foreign Application Priority Data
Jul. 15, 2021 (HU) P2100269

(51) Int. Cl. G01N 33/543; G01N 33/58

(52) U.S. Cl. CPC G01N 33/54386; G01N 33/58

(57) ABSTRACT

A device for determining one or more of multiple analytes in a sample liquid includes a detection surface having thereon a microarray of elements, comprising: a plurality of binding elements immobilized by insoluble binding at discrete locations on the detection surface and a plurality of dissolvable elements immobilized by soluble binding at other discrete locations on the detection surface. The elements being arranged in the microarray such that at least a portion of the dissolvable elements is located closer to the center of the microarray and/or at least a portion of the dissolvable elements is located closer to the periphery of the microarray than each of the binding elements. Alternatively, the dissolvable and immovable elements may be provided similarly or mirrored in areas which may be duplicated.

### CROSS-REFERENCE TO RELATED APPLICATIONS

This application is a national phase under 35 U.S.C. § 371 of PCT International Application No. PCT/IB2022/056420 which has an International filing date of Jul. 12, 2022, which claims priority to Hungarian Application No. P2100269, filed on Jul. 15, 2021 in the Hungarian Intellectual Property Office, the entire contents of each of which are hereby incorporated by reference.

### BACKGROUND OF THE INVENTION

### 1. Field of the Invention

The present invention relates to a device and a system for the simultaneous detection of multiple analytes in the same liquid sample as well as a method for carrying out an assay for said simultaneous detection.

In particular, the present invention relates to a device for use in the determination of one or more of multiple analytes in a liquid sample, said device comprising a detection surface having thereon a microarray of elements, comprising a plurality of binding elements immobilized by insoluble binding at discrete locations on the detection surface, wherein said binding elements include one or more of different analyte related binding elements; and a plurality of dissolvable elements which are immobilized by soluble binding at other discrete locations on the detection surface.

### 2. Related Art

Multiplexed assays have been commonly used for more than a decade and have already found many important uses including clinical diagnosis, gene expression analysis, high throughput screening, drug discovery, and environmental, veterinary, forensic and food sciences. A multiplexed assay is any assay where multiple targets (also known as “analytes”) can be measured in a single sample. Multiplexed assays can be performed using a variety of methods, including microarray, bead-based formats, and microfluidics.

The microarray type assays generally comprise a collection of multiple binding elements immobilized as dots at discrete locations in a microarray on a detection surface. These binding elements typically include a plurality of replicate target related binding elements, each of the plurality being used in the detection of a corresponding different one of the multiple analytes in the single sample by measuring an analyte-related signal, usually an optical signal, the intensity of which typically reflects the concentration of a one of the multiple analytes in the sample.

Reasons for current limitations of microarray type assay measurements vary, but major contributors include variation in printing of the immobilized binding elements, which printing can vary element-to-element, well-to-well, slide-to-slide, and run-to-run; variation in the conjugation of the binding elements to surfaces; variation in surface chemistry that occurs in wells and on detection surfaces; variation in reagent mixing and handling, particularly when assays are performed by hand. In particular, variation occurs between days and between operators; and variation in detection instruments—including day-to-day variation between instruments as well as variation of a single instrument. There is therefore a need to detect analytes, particularly analytes present in low concentrations, quickly, accurately and reproducibly.

Microarrays and similar technologies may be seen in WO 2009/039170, US2018/0154353A1, U.S. Pat. Nos. 8,298,834, 5,356,785, ‘Antibody Microarray Immunoassay for Simultaneous Quantification of Multiple Mycotoxins in Corn Samples”; Xian Zhang et al.; Toxins, 15 Oct. 2018; pages 1-13, in and “Rapid and sensitive detection of mycotoxins by advanced and emerging analytical methods: review; Jyoti Singh et al.; Food Sci Nutri.; Z5 Mar. 2020.

### BRIEF SUMMARY OF THE INVENTION

The invention relates to a device according to claim 1.

The elements may comprise assay elements which may include dissolvable target detection reagents, preferably provided as at least a portion of the dissolvable elements which are present on the detection surface as spots or dots. By introducing dissolvable elements which comprise target detection reagents the need for the separate addition of assay specific reagents is minimized or removed. Moreover, separating the dissolvable elements, preferably dissolvable spots or dots, from the immobilized elements, preferably fixed spots or dots in a geometric advantageous manner obtains improved control of factors, for example kinetics influenced assay performance, that negatively impact test variations. This can be performed in a single device, such as a microarray, by, for example, exposing the dissolvable elements, preferably spots or dots, to the liquid sample prior to exposing to it the fixed capture dots. Furthermore, in an embodiment by using dot location based on geometric considerations, the microarray can be designed such that only dissolvable elements are located where liquid sample will be dispensed onto the microarray thus reducing the risk of fixed elements being dislocated by the flow pressure as the liquid sample is dispensed, and/or the microarray can be designed so that replicates of elements have close to identical physical exposures to, for example, the sample, assay reagents and other assay liquids such as wash buffer, during any or all of liquid sample application, incubation, and washing. This is obtained by using a non-crossover non-scattering location of the fixed elements in the microarray(s) and accordingly, the need to form aliquots of the sample and introducing each aliquot to separate microarrays, for example in separate wells or containers, in order to obtain a reliable measurement is waived. This saves steps and complexity in performing the assay along with ensuring that the analyte binding reactions experience substantially the same reaction conditions.

In an embodiment of the device the one or more analyte-related binding elements comprise respective analyte analogues, to which the dissolvable target detection reagent is capable of binding.

In an embodiment of the device of the invention the immobilized binding elements include reference binding elements and the assay elements additionally or separately include dissolvable reference reagents, preferably provided as at least a portion of the dissolvable elements which are present on the detection surface as spots or dots. The dissolvable reference reagents are adapted to bind to the reference binding elements and which, when bound, produce a detectable reference signal, and wherein each dissolvable reference reagent is located either closer to the center of the microarray or to the periphery of the microarray than the reference binding element to which it is adapted to bind, optionally wherein a portion of the dissolvable reference reagents is located closer to the center of the microarray and another portion of the dissolvable reference reagents is located closer to the periphery of the microarray than the reference binding element to which they are adapted to bind. In this manner the analyte-related signals measured from the analyte-related binding reactions may be standardized and normalized by the reference signals coming from the bound reference reagents, making the assay self-referencing upon a single introduction of the liquid sample to the microarray. Accordingly, the need to form aliquots of the liquid sample and introducing each aliquot to separate microarrays, for example in separate wells or containers, in order to obtain the same self-referencing, is waived, as is the need of introducing the reference reagent separately. This saves steps and complexity in performing the assay along with ensuring that the target binding reactions and the reference binding reactions experience substantially the same reaction conditions.

Thus, the device may further comprise, on the detection surface, a plurality of dissolvable reference elements which are dissolvable upon applying the liquid sample to the detection surface, the dissolvable reference elements comprising one or more reference components. These dissolvable elements may be attached to the detection surface by soluble binding and/or at fourth discrete locations on the detection surface.

In that or another embodiment, the device further comprises, on the detection surface, a plurality of immobilized reference elements, which may be immobilized by insoluble binding and/or at fifth discrete locations on the detection surface, each immobilized reference element comprising one or more reference binding components each being configured to attach to one or more of the multiple analytes and/or one or more of the reference components.

In an embodiment of the device of the invention the reference binding elements are located relative to the analyte related binding elements to provide the detectable reference signal that is subject to the same device and measurement conditions variations as the detectable analyte-related signal. In this manner the relative geometric arrangement of the insolubly bound reference binding elements and the insolubly bound analyte related binding elements may provide increased accuracy and precision of the assay.

In some embodiments the detectable reference signal and the detectable analyte-related signal is each an optically detectable signal. Usefully, the immobilized reference binding elements are positioned on the detection surface in order to provide the detectable reference signal or signals for use in mitigating the effects of optical distortion on the determination, preferably wherein the reference binding elements are positioned on the detection surface to take into account the optical properties of one or both of an optical detection device and the detection surface thereby improving one or both a standardization and a calibration signal.

In an embodiment of the device of the invention each analyte-related binding element comprises an analyte analogue which competes with the analyte for binding to the dissolvable target detection reagent upon applying a liquid sample comprising the analyte such that dissolvable target detection reagents not bound to the analyte, bind to the analyte-related binding elements, wherein a higher concentration of analyte in the liquid sample results in a lower detectable analyte-related signal level and a lower concentration of analyte in the liquid sample results in a higher detectable analyte-related signal level.

In an embodiment of the device of the invention each analyte-related binding element for an analyte is capable of binding the respective analyte and an analyte analogue and each dissolvable target detection reagent comprises an analyte analogue capable of binding to the analyte-related binding element and also comprises a label capable of providing the analyte-related signal, wherein each dissolvable target detection reagent competes with the analyte in the liquid sample for binding to the analyte-related binding element, wherein a higher concentration of analyte in the liquid sample results in a lower detectable analyte-related signal level and a lower concentration of analyte in the liquid sample results in a higher detectable analyte-related signal level.

In a further embodiment of the device of the invention each analyte-related binding element is capable of binding the analyte and is also capable of binding an analyte analogue, the dissolvable target detection reagents comprise an analyte analogue that is conjugated to a molecule that can specifically bind to a binding agent to provide a labelled target compound and the device, preferably the microarray also comprises a labelled binding compound which comprises the binding agent and a label molecule for providing the analyte-related signal and is capable of binding the dissolvable target detection reagent, and wherein upon binding of the dissolvable target detection reagent to the analyte-related binding element the labelled binding compound also binds to the dissolvable target detection reagent, wherein the labelled target compound competes with the analyte for binding to the analyte-related binding element for the same analyte so that a higher concentration of analyte in the liquid sample results in a lower detectable analyte-related signal level and a lower concentration of analyte in the liquid sample results in a higher detectable analyte-related signal level.

In an embodiment of the device of the invention both the analyte-related binding elements and the dissolvable target detection reagents bind simultaneously and specifically to the same analytes at different binding sites. For example, in the case of proteins the different binding sites may be different epitopes; in the case of complex analytes the different binding sites may be different ligands; and in the case of nucleic acid analytes the different binding sites may be different sequence segments.

In an embodiment of the invention the dissolvable target detection reagents are labelled binding compounds and upon binding of the analytes in the liquid sample to the analyte-related binding elements, the labelled binding compounds also bind to the bound analytes and thereby to the analyte-related binding elements so that a higher concentration of analyte in the liquid sample results in a higher detectable analyte-related signal level and a lower concentration of analyte results in a lower detectable analyte-related signal level.

In some of the embodiments the immobilized binding elements include reference binding elements and the dissolvable elements include dissolvable reference reagents adapted to bind to the reference binding elements to provide the detectable reference signal, and wherein each dissolvable reference reagent is located either closer to the center of the microarray or to the periphery of the microarray than the reference binding element to which it is adapted to bind, optionally wherein a portion of the dissolvable reference reagents is located closer to the center of the microarray and another portion of the dissolvable reference reagents is located closer to the periphery of the microarray than the reference binding element to which they are adapted to bind.

In an embodiment one or both the immobilized binding elements and the dissolvable elements are added to the detection surface in the form of spots having defined boundaries, such as dots (i.e. round, preferably essentially circular shaped areas having a boundary). Preferably dots are applied by placing drops comprising the elements in a solvent. In a particular embodiment the elements and/or the surface is/are functionalized to establish insoluble binding in case of immobilized elements.

In a particular embodiment optically detectable label is added to pre-defined locations on the detection surface in order to indicate orientation for the correct identification of the different spots, preferably dots upon analysis of the optical signals produced by the spots, preferably dots.

In an embodiment of the device of the invention a portion of the dissolvable target detection reagents is located closer to the center of the microarray and another portion of the dissolvable target detection reagents is located closer to the periphery of the microarray than the analyte-related binding element for the same analyte. It may be desired that all first discrete locations are closer to the centre than any of the second discrete locations.

In an embodiment of the device of the invention at least part of the dissolvable elements are located in an area of the detection surface comprising the center of the microarray, so that when the liquid sample is applied to the center a “later” assay component located relatively closer to the periphery of the microarray is dissolved differently time-wise to the “early” components that are located relatively closer to the centre of the microarray.

In a variant, dissolvable elements are co-located at the same spot or dot. Thereby a more even distribution of said dissolvable elements may be obtained.

In an embodiment of the device of the invention the periphery of the detection surface comprises only dissolvable elements, preferably dots so that the periphery becomes free of said elements after the addition of the liquid sample to the detection surface. In this device there is a reduced risk that reflections from the side-walls would distort the optical reading of dots.

In one embodiment:
the dissolvable elements comprise a first group of dissolvable elements and a second group of dissolvable elements,
the first discrete locations of all dissolvable elements of the first group are positioned, on the detection surface, closer to the centre than the second discrete locations of any of the immobilized binding elements and
the second discrete positions of all of the immobilized binding elements are positioned, on the detection surface, closer to the centre than the first discrete positions of any of the dissolvable elements of the second group.

Thus, the first and second group are positioned on either side of the locations of the immobilized binding elements. This may be taken advantage of e.g. during a step of dispending the liquid sample on to the detection surface. The flow and flow direction of the liquid sample may be directed first to the first group, then to the immobilized binding elements and then to the second group. Thus, the contents of the liquid sample may, due to the dissolvable elements, vary over the detection surface, which may be advantageous.

Then, the device may further comprise additional immobilized elements each positioned at a third discrete location, wherein the first discrete locations of all dissolvable elements of the second group are positioned, on the detection surface, closer to the centre than any of the third discrete locations.

In addition, or alternatively, each second discrete location may be positioned so that a first straight line through the centre and the pertaining second discrete location has an angle of no more than 20 degrees, such as no more than 15 degrees, such as no more than 10 degrees, such as no more than 5 degrees, to a second straight line through the centre and a first discrete location. In this manner, if individual portions of the liquid sample flows along straight lines, the positions of the discrete locations on the device may ensure that the portion of the liquid sample arriving at a particular second discrete location has been sufficiently close to a pertaining first discrete location that portions or contents of the dissolvable element at that first discrete location may be entrained in the liquid sample when arriving at the second discrete location. The straight lines often extend to an edge of the detection surface, which may be a predetermined area of a larger surface such as in the situation where the same surface has a plurality of detection surfaces.

In a variant of the invention each of the one or more dissolvable target detection reagents is capable of binding an analyte (i.e. being an analyte detection reagent), preferably each being adapted to bind to a different analyte.

In a particular variant of embodiments of the invention the dissolvable elements include functionalized particles.

Another aspect of the invention relates to a device for use in the determination of one or more of multiple analytes in a liquid sample, said device including a detection surface for exposure to the liquid sample and having thereon a number of elements comprising:
a plurality of dissolvable elements which are dissolvable upon applying the liquid sample to the detection surface, said dissolvable elements being attached to the detection surface, such as by soluble binding, at first discrete locations on the detection surface, the dissolvable elements comprising one or more first components, and
a plurality of immobilized elements immobilized, such as by insoluble binding, at second discrete locations on the detection surface, each immobilized element comprising one or more binding components each being configured to attach to one or more of the multiple analytes and/or one or more of the first components,
   wherein the detection surface has a centre, where a plurality of non-overlapping first areas exists on the detection surface, each first area being delimited at least partly by two straight lines through the centre, where each first area has the same amount of dissolvable elements and immobilized elements.

In this context, the same amount may be the same volume, concentration, number of dots, size of the dots, same number of a particular type of agent, reactant, or the like.

Thus, within the first areas, the same analysis may be made, as each has the same amount of the dissolvable and immobilized elements. Multiple areas with the same features may then be used for increasing the precision or the accuracy of the results.

In one embodiment, the two straight lines, for at least two of the non-overlapping first areas, define at least substantially the same angle between them, where “at least substantially” may mean that a larger angle is no more than 110% of a smaller angle.

Preferably, at least two of the non-overlapping first areas have at least substantially the same areas, such as where a larger area is no more than 110% of a smaller area.

Preferably, at least two of the non-overlapping first areas have the same number of first discrete locations. Then, the first discrete locations in the at least two non-overlapping first areas may define sets of first discrete locations, each set having one first discrete location in each of the at least two non-overlapping first areas, wherein the first discrete locations of each set are at least substantially at the same distance to the centre. In this context, “at least substantially” may mean that a larger distance of a set is no more than 110% of a smaller distance of the set.

A set represents one first discrete location from each area. When the distances of a set are identical, the two areas have a dissolvable element at the same distance from the centre.

It is preferred that at least two of the non-overlapping first areas have the same number of second discrete locations. Then, the second discrete locations in the at least two non-overlapping first areas may define sets of second discrete locations, each set having one second discrete location in each of the at least two non-overlapping first areas, wherein the second discrete locations of each set are at least substantially at the same distance to the centre. In this context, “at least substantially” may mean that a larger distance of a set is no more than 110% of a smaller distance of the set.

In a preferred embodiment, the number of first and second discrete locations are identical in the at least two non-overlapping first areas.

It may be desired that two of the at least two non-overlapping first areas are positioned on opposite sides of the centre. In this context, the areas are opposite if a straight line exists through the centre and each of the two non-overlapping first areas.

In one embodiment, the first and second discrete locations of the two non-overlapping first areas are mirrored around the centre. Mirroring will mean that straight lines exist through the centre and pairs of first or second discrete locations—where the distances from the centre to the pertaining first/second discrete locations are the same.

In one embodiment, in at least one of the non-overlapping first areas, all first discrete locations are closer to the centre than any of the second discrete locations. The advantages of this are described further above.

Then, in the at least one of the non-overlapping first areas:
the dissolvable elements comprise a first group of dissolvable elements and a second group of dissolvable elements,
the first discrete locations of all dissolvable elements of the first group are positioned, on the detection surface, closer to the centre than the second discrete locations of any of the immobilized binding elements and
the second discrete positions of all of the immobilized binding elements are positioned, on the detection surface, closer to the centre than the first discrete positions of any of the dissolvable elements of the second group.

Then, the device could further comprise, in the at least one of the non-overlapping first areas, additional immobilized elements each positioned at a third discrete location, wherein the first discrete locations of all dissolvable elements of the second group are positioned, on the detection surface, closer to the centre than any of the third discrete locations.

In addition to the first areas, the device may comprise two second, non-overlapping areas, which are non-overlapping also with the first non-overlapping areas, where the first and second non-overlapping areas are not the same, such as if the first and second areas have:
different numbers of first discrete locations,
different numbers of second discrete locations,
different distances from the first discrete locations to the centre,
different distances from the second discrete locations to the centre,
different areas,
different angles between the two straight lines at least partly delimiting the pertaining area,
different first components of the dissolvable elements, and/or
different binding components of the immobilized elements.

Then, in one embodiment, the number of first and second discrete locations are identical in the two non-overlapping second areas.

Also, or alternatively, the two non-overlapping second areas can be positioned on opposite sides of the centre. Then, the first and second discrete locations of the two non-overlapping second areas may be mirrored around the centre.

In general, the device may further comprise elements configured to guide the liquid sample along at least substantially straight paths from the centre and radially therefrom. This could be ridges or the like ensuring that the same portion of the liquid sample is passed from one or more desired dissolvable elements to desired immobilized elements. By providing liquid guides, different portions of the liquid may be used for different purposes, such as the determination of different agents.

In one embodiment, the detection surface is plane. In this manner, liquid may be transported over the detection surface by rotation, vibration or the like.

In another embodiment, the detection surface is cone-shaped, so that gravity may assist in the transport of the liquid over the detection surface.

In general, the device may further comprise, on the detection surface, a plurality of dissolvable reference elements which are dissolvable upon applying the liquid sample to the detection surface, the dissolvable reference elements comprising one or more reference components. These dissolvable elements may be attached to the detection surface by soluble binding and/or at fourth discrete locations on the detection surface.

In general, the device may further comprise, on the detection surface, a plurality of immobilized reference elements, each immobilized reference element comprising one or more reference binding components each being configured to attach to one or more of the multiple analytes and/or one or more of the reference components. The immobilized reference elements may be immobilized by insoluble binding and/or at fifth discrete locations on the detection surface.

Preferably, the reference binding elements are located on the detection surface relative to the binding components, also called analyte related binding elements, to provide the detectable reference signal that is subject to the same device and measurement conditions variations as the detectable analyte related signal.

In general, the binding components include one or more of different analyte-related binding components for each analyte, any or each of said analyte-related binding components being adapted to attach to a first component and/or an analyte. Preferably this is to an extent that is related to the concentration of the analyte in the liquid sample. Thus, the amount of binding may relate to the concentration. Then, when attached the analyte may be capable of, itself or an agent attached thereto, producing a detectable analyte-related signal for use in making a determination of the presence or amount of the analyte in the liquid sample.

The one or more analyte-related binding components may comprise respective analyte analogues, to which the one or more first components is capable of attaching.

Also, the detectable signal is preferably an optically detectable signal. The binding elements may be located in the microarray on the detection surface in a manner so as to avoid or reduce optical distortion, preferably where the reference binding elements are located in the microarray on the detection surface to take into account the optical properties of one or more of an optical detection device and the detection surface thereby improving one or both of a standardization and a calibration signal.

In general, each binding component may be configured to attach to an analyte or an analyte analogue and a first component and may further comprise a label capable of generating an output signal.

In general, each binding component may be capable of attaching to:
an analyte,
an analyte analogue and
one of the one or more first components, comprising an analyte analogue that is conjugated to a molecule that can specifically attach to the pertaining binding component.

Preferably the device or microarray also comprises a labelled binding compound/component which comprises the binding agent and a label molecule for providing the analyte-related signal and which is capable of binding the dissolvable first component of target detection reagent, and wherein upon binding of the dissolvable target detection reagent to the analyte-related binding element the labelled binding compound also binds to the dissolvable target detection reagent.

In general, preferably both the (analyte-related) binding components and the first components (and/or dissolvable target detection reagents) attach simultaneously and specifically to the same analytes at different binding sites, or to different ligands. Then, the first components (and/or dissolvable target detection reagents) are preferably labelled binding compounds and wherein, upon binding of the analytes in the liquid sample to the (analyte-related) binding components, where the labelled binding compounds also attach to the attached analytes and thereby to the (analyte-related) binding components.

In general, a portion of the first components is preferably located closer to the centre and another portion of the first components is located closer to a periphery of the detection surface than the (analyte-related) binding component for the same analyte.

In general, at least some of the dissolvable elements are preferably located in the centre of the detection surface, so that preferably, when the liquid sample is applied to the centre a later assay component located relatively closer to the periphery of the microarray is dissolved differently timewise to early components that are located relatively closer to the centre of the detection surface, and preferably dissolvable elements are co-located.

In one embodiment, a periphery of the detection surface comprises only dissolvable elements so that it becomes free of said elements after the addition of the sample liquid on the detection surface so that there is a reduced risk that reflections from the side walls would distort the optically detectable signal.

In that or another embodiment, the dissolvable elements include functionalized magnetic particles.

Yet another aspect of the invention relates to a method for determining one or more of multiple analytes in a liquid sample using a device according to any of the preceding aspects, said method comprising the steps of:
a) applying a liquid sample to or at the centre of the detection surface,
b) guiding the liquid sample to the dissolvable elements to at least partly dissolve the dissolvable elements to release the one or more first components into the liquid sample,
c) guiding the liquid sample with the one or more first components to the immobilized elements to have the one or more binding components attach to the one or more of the multiple analytes and/or the one or more of the first components,
d) detecting a signal output by
   the one or more binding components attached to the one or more of the multiple analytes and/or the one or more of the first components and/or
   the one or more of the multiple analytes and/or the one or more of the first components attached to the one or more binding components; and
e) determining, based on the signal, the presence or amount of at least one of the multiple analytes in the liquid sample.

Naturally, all aspects of the invention, all embodiments, situations, means and steps may be interchanged between aspect of the invention. Thus, a number of the features of this aspect of the invention have been described further above in relation to other aspects of the invention.

In one embodiment, at least one of the guiding steps comprises rotating the disc around an axis provided through the centre. In that or another embodiment, during at least one of the guiding steps, the detection surface is vibrated.

In one embodiment, at least a portion of the first discrete locations is located closer to a centre of the detection surface than each of the second discrete locations and wherein the guiding steps comprise guiding the liquid sample in a direction away from the centre.

In one embodiment, the detection surface has a centre, where a plurality of non-overlapping first areas exists on the detection surface, each first area being delimited at least partly by two straight lines through the centre, where each first area has the same amount of dissolvable elements and immobilized elements and wherein the guiding steps comprise guiding the liquid sample at least substantially simultaneously over the at least two non-overlapping areas.

In one embodiment, the guiding steps comprise essentially guiding different portions of the liquid sample inside different/respective first non-overlapping areas.

In one embodiment, the applying step comprises maintaining the applying of liquid sample until liquid sample has reached all first and second discrete locations.

One embodiment further comprises the step of, between the guiding steps and the detection step, flowing a second liquid over the detection surface and the first and second discrete locations. This may be a washing step, such as aiming at removal of non-bound first components and/or liquid sample.

In one embodiment, the guiding step b) comprises attaching an analyte of the liquid sample to a first component of a dissolvable element. Then, the guiding step c) could comprise attaching the analyte or the first component to a binding component of an immobilized element.

In one embodiment, the device further comprises, on the detection surface, a plurality of dissolvable reference elements which are dissolvable upon applying the liquid sample to the detection surface, the dissolvable reference elements comprising one or more reference components and

wherein the detecting step comprises detecting a second signal output by the one or more reference components. In this case, said dissolvable elements could be attached to the detection surface by soluble binding and/or at fourth discrete locations on the detection surface.

In one embodiment, the device further comprises, on the detection surface:
a plurality of dissolvable reference elements which are dissolvable upon applying the liquid sample to the detection surface, the dissolvable reference elements comprising one or more reference components and
a plurality of immobilized reference elements and having one or more reference binding components,
   the method comprising the additional guiding step of guiding liquid sample from the dissolvable reference elements to the immobilized reference elements to the one or more reference binding components attach to one or more of the multiple analytes and/or one or more of the reference components, and
   wherein the detecting step comprises detecting a second signal output by the binding components and/or the one or more analytes and/or reference components attached to the binding components.

As mentioned above, said dissolvable elements could be attached to the detection surface by soluble binding and/or at fourth discrete locations on the detection surface.

Also, the immobilized reference elements could be immobilized by insoluble binding and/or at fifth discrete locations on the detection surface.

In one embodiment, the detecting step comprises launching first radiation toward the detecting surface and detecting second radiation received from:
the one or more binding components attached to the one or more of the multiple analytes and/or the one or more of the first components
the one or more of the multiple analytes and/or the one or more of the first components attached to the one or more binding components and/or
a marker attached to the one or more binding components, the one or more of the multiple analytes and/or the one or more first components.

Then, the second radiation could be generated by:
the one or more binding components attached to the one or more of the multiple analytes and/or the one or more of the first components and/or
the one or more of the multiple analytes and/or the one or more of the first components attached to the one or more binding components absorbing a portion of the first radiation and transmitting/reflecting/scattering/outputting, as the second radiation, another portion of the first radiation.

Also, or alternatively, the second radiation could be generated by:
the one or more binding components attached to the one or more of the multiple analytes and/or the one or more of the first components and/or
the one or more of the multiple analytes and/or the one or more of the first components attached to the one or more binding components, absorbing a portion of the first absorbing a portion of the first radiation and emitting, as the second radiation, fluorescence.

In a particular variant of embodiments of the invention the dissolvable elements include functionalized magnetic particles.

In an embodiment the device having the detection surface is suitable for centrifugation to increase sedimentation of the dissolvable elements and thereby enhance contact with the detection surface, which facilitates a faster and more efficient binding of the targets to be detected to the analyte-related binding elements.

In an embodiment centrifugation facilitates sedimentation of functionalized particles.

In a particular embodiment the device comprises a container or a series of containers the bottom of which has or comprises the detection surface. The container or series of containers is preferably suitable for centrifugation. The series of containers may be an array of containers, optionally in a matrix form. In a particular embodiment the container(s) is/are well(s).

In an embodiment of the device the detection surface is a circular plane.

In an embodiment said determination is qualitative. In this embodiment the detectable analyte-related signal indicates the presence or absence of the analyte in the sample relative to a predetermined concentration threshold value.

In a particular embodiment the analyte-related binding elements are immobilized to a surface with a spacer molecule that extends the distance of the binding agent from the surface.

In a particular embodiment the analyte-related binding elements are immobilized to a surface without a spacer molecule that extends the distance of the binding agent from the surface.

In particular embodiments, the analyte-related binding elements comprise an analyte analogue selected from the group consisting of nucleic acids and nucleic acid analogues, mono- or polyclonal-antibodies or their fragments, peptides, enzymes, aptamers, synthetic peptide structures, membrane-bound proteins and proteins isolated from a membrane, including receptors, their ligands, antigens for antibodies, histidine-tag components and their complex forming partners, and cavities generated by chemical synthesis, for hosting molecular imprints, are deposited, or wherein whole cells, cell components, cell membranes or their fragments are deposited as biological or biochemical or synthetic binding elements.

In further particular embodiments, the analyte-related binding elements are capable of binding an analyte selected from the group consisting of nucleic acids, mono- or polyclonal-antibodies, peptides, enzymes, membrane-bound proteins and proteins isolated from a membrane, receptors, ligands, antigens for antibodies, cell components, cell membranes or their fragments, toxins, hormones, cytokines, cells and cellular parts like compartments, viruses and viral parts, etc.

In particular embodiments the dissolvable target detection reagents are labelled binding molecules which are selected from or which comprise one or more moieties selected from the group consisting of nucleic acids and nucleic acid analogues, mono- or polyclonal-antibodies or their fragments, peptides, enzymes, aptamers, synthetic peptide structures, soluble membrane-bound proteins and proteins isolated from a membrane, including receptors, their ligands, antigens for antibodies, protein tags, e.g., GST-tag, peptide tags, e.g., histidine- or FLAG-tag, and components and their complex forming partners, avidin, streptavidin, neutravidin, biotin, and cavities generated by chemical synthesis, for hosting molecular imprints, are deposited, or wherein whole cells, cell components, cell membranes or their fragments are deposited as biological or biochemical or synthetic binding elements.

In particular embodiments the detectable signal is an optically detectable signal.

In particular embodiments the label that provides a detectable signal is an optical signal producing molecule selected from the group consisting of chromogenic, luminescent, chemi-luminescent or fluorescent molecules, preferably one or more of fluorescent proteins, small molecules fluorescent dyes, quantum dots, fluorogenic ligands and fluorogen-activating proteins, and/or wherein the label that provides a signal comprises one or more of gold, silver, polystyrene, or latex, silica or magnetic particles, or nanoparticles, and/or wherein the label that provides a signal comprise one or more of chromophores produced by activities of chromogenic enzyme-substrate systems (e.g. HRP-DAB/HRP-TMB/OPD/ABTS, AP-AEC/pNpp) and (if needed) different signal amplification systems (e.g. avidin/streptavidin/neutravidin-biotin).

In a preferred embodiment the detection surface further comprises laterally separated measurement areas, said laterally separated measurement areas being generated by laterally selective deposition of biological, biochemical or synthetic, binding elements, by using a method comprising inkjet, contact or non-contact spotting, including mechanical spotting using a pen, pin or capillary, micro contact printing, fluidic contacting of the measurement areas with the biological, biochemical or synthetic binding elements upon their supply in parallel or crossed micro channels, upon application of pressure differences or electric or electromagnetic potentials, and photochemical or photolithographic immobilization methods. Such binding elements may be immobilized binding elements or dissolvable elements, provided that such elements are suitable for binding of other elements or analytes.

In further particular embodiments of the invention the detection surface is selected from a group consisting of: bottom of wells of plastic or membrane-based 6-, 24-, 78-, 96-, 386-, or 1536-well microtiter plates, strips of wells, breakable strips; and single well inserts.

In an embodiment said dissolvable reference elements and/or analyte-related binding elements are immobilized at different measurement areas of the detection surface at different known concentrations such that the reference signals and/or analyte related signals obtained therefrom may be used to establish standardization and/or a calibration curve for the analytes.

In a further aspect the invention relates to a measurement system for the simultaneous determination of one or more analytes comprising:
a device according to any of the embodiments as defined above or as defined in the appended claims wherein the analyte-related signal is an optically detectable signal;
at least one spatially-resolved detector for detecting the intensity and location of the light signals; and
an analysis unit, consisting of the hardware and software for the analysis of the detected signals to determine the presence or amount of an analyte in a liquid sample.

In a preferred embodiment the optically detectable signal is evolved upon illumination and the system also comprises at least one illumination light source that is adequate for inducing specific optical signals from the said labelled molecules.

In a particular embodiment the optical system is configured to identify the orientation of the assay by the detection of an optically detectable label which is added to pre-defined locations on the detection surface.

In a further aspect the invention relates to a method for determining one or more of multiple analytes in a liquid sample, using a device according to a previous aspect of the invention.

In an embodiment or variant of the method for determining one or more of multiple analytes in a liquid sample a device according to any one of the embodiments of the invention or a device as described herein is used, said method comprising the steps of:
applying a liquid sample to the detection surface to dissolve the dissolvable target detection reagents and the dissolvable reference reagents, when present, into the liquid sample, whereby at least a portion of the dissolvable target detection reagents binds to analyte and/or to analyte-related binding elements for the given analyte or at least a portion of the dissolvable target detection reagents binds to analyte and another portion to analyte related binding elements for the given analyte;
optionally removing the non-bound part of the liquid sample by washing;
detecting the analyte-related signal and optionally the reference signal; and
determining the presence or amount of analyte in the liquid sample from the detected analyte-related signal.

In an embodiment an amount, e.g. quantity or concentration is determined. In this embodiment the level of the detectable analyte-related signal preferably correlates with the concentration of the analyte in the liquid sample or with a quantity derived from the concentration of the analyte in the sample. A quantity is a mathematical quantity derived by a function calculated from the detectable analyte-related signal level or derived by a calibration curve or a physical quantity derived from the detectable analyte-related signal level related to the concentration level by a physical transformation by an experimental or mathematical means. In a variant of the assay the detectable analyte-related signal level directly correlates with the concentration of the analyte in the liquid sample. In a variant of the assay the detectable analyte-related signal level inversely correlates with the concentration of the analyte in the sample. In an embodiment the detectable analyte-related signal level is directly proportional with the concentration of the analyte in the sample or with a quantity derived from the concentration of the analyte in the sample, or the detectable analyte-related signal level correlates via a calibration curve to the concentration of the analyte in the sample. The calibration curve or physical transformation may be established in a conventional manner from measurements of analyte-related signals obtained using liquid samples having known concentrations of analyte and establishing therefrom a relationship linking detected analyte-related signal level to amount of analyte. The relationship is then applied to a detected analyte-related signal level from a liquid sample containing an unknown amount of analyte in order to obtain a determination of the amount of analyte in the liquid sample.

In a preferred embodiment the method comprises washing to remove the non-bound part of the liquid sample.

In a preferred embodiment of the method at least a portion of the dissolvable target detection reagents binds to analyte present in the liquid sample and/or at least a portion of the dissolvable target detection reagents binds to analyte analogues, said analyte analogues being bound to analyte related binding elements for the given analyte, wherein said analyte is either insolubly bound to the analyte related binding element or binds to the analyte related binding element upon applying the liquid sample.

In a preferred embodiment of the method the dissolvable elements include dissolvable reference reagents adapted to bind to the reference binding elements as defined above, and wherein upon applying the liquid sample on the surface each dissolvable reference reagent binds to the reference binding element to which said element is adapted to bind, wherein said bound reference reagent results in a detectable reference signal, and said method also comprising: detecting the reference signal; determining a deviation of the detected reference signal from a predetermined reference signal; and adjusting the analyte-related signal in dependence on the determined deviation.

Preferably the reference signal is subject to same device variations as the analyte-related signal. In this manner the analyte-related signal may be standardized and normalized by the reference signals making the assay self-referencing upon a single introduction of the liquid sample to the microarray.

Preferably in the method of the invention a device is used, wherein both the reference signal and the analyte-related signal are optically detectable signals and the immobilized binding elements are located on the detection surface so as to avoid or reduce optical distortion, preferably wherein the reference binding elements are located on the detection surface to take into account the optical properties of one or more of the optical detection device, the detection surface and the container, thereby improving one or both of a standardization and a normalization of the analyte-related signal.

In a preferred embodiment of the method a device is used wherein each analyte-related binding element comprises an analyte analogue and thus the analyte-related binding element competes with the analyte for the dissolvable specific target detection reagent upon applying a liquid sample comprising the analyte, and wherein at least a portion of the dissolvable target detection reagents bind the analyte if present in the liquid sample, wherein optionally the bound analyte targets are removed by washing, and wherein dissolvable target detection reagents not bound to the analyte, binds to the target analyte-related binding elements, such that a higher concentration of analyte in the liquid sample results in a lower analyte-related signal level detectable from the respective analyte-related binding elements, and a lower concentration of analyte in the liquid sample results in a higher analyte-related signal level detectable from the same analyte-related binding elements.

In an embodiment of the method according to the invention a device is used, wherein each analyte-related binding element for an analyte is capable of binding the respective analyte and analyte analogue and each dissolvable target detection reagent comprises an analyte analogue and is capable of binding to the analyte-related binding element and also comprises a label capable of providing a signal upon binding,

wherein each dissolvable target detection reagent competes with the analyte in the liquid sample for binding to the analyte-related binding element, such that a higher concentration of analyte in the liquid sample results in a lower analyte-related signal level and a lower concentration of analyte in the liquid sample results in a higher analyte-related signal level, and determining therefrom the amount of analyte in the liquid sample based on a calibration generated from analyte-related signals obtained from a plurality of liquid samples containing different known amounts of analytes.

In an embodiment of the method a device is used wherein each analyte-related binding element is capable of binding the analyte and is also capable of binding the analyte analogue; the dissolvable target detection reagents comprise an analyte analogue that is conjugated to a molecule that can specifically bind to a binding agent that is conjugated to a labelling molecule to provide a labelled target compound and the device, preferably the microarray, also comprises a labelled binding compound which consists of the said binding agent and a label molecule for providing the analyte-related signal and is capable of binding a target detection reagent for which it is adapted to bind, and wherein upon binding of the target detection reagent to the analyte-related binding element the labelled binding compound also binds to the target detection reagent; wherein the labelled target compound competes with the analyte for binding to the analyte-related binding element for the same analyte such that a higher concentration of analyte in the liquid sample results in a lower analyte-related signal level and a lower concentration of analyte in the liquid results in a higher analyte-related signal level, and wherein preferably determining the amount of analytes in the liquid sample comprises determining the amount of the analyte related binding elements binding labelled target compounds, wherein from this and the total amount of analyte related binding elements on the assay surface the amount of analyte related binding elements binding analyte is determined from which the level of analyte in the liquid sample is assessed.

In a preferred embodiment of the method a device is used wherein both the analyte-related binding elements and the dissolvable target detection reagents bind simultaneously and specifically to the same analytes at different binding sites, or to different ligands, preferably wherein the dissolvable target detection reagents are labelled binding compounds and wherein upon binding of the labelled binding compounds to the analytes that are bound to the analyte-related binding elements to which they are adapted to bind that the signal that evolves is related to the said analyte concentration, such that a higher concentration of analyte results in a higher analyte-related signal level and a lower concentration of analyte results in a lower analyte-related signal level and wherein preferably assessing the level of analytes in the liquid sample comprises determining the amount of labelled binding compounds bound to the analytes and thereby to the analyte related binding elements and from this the level of analyte in the liquid sample is assessed.

In a particular embodiment of the method of the invention a device as defined herein, in particular the device as described hereinafter, is used, being a device wherein a portion of the dissolvable target detection reagents is located closer to the center of the microarray and another portion of the dissolvable target detection reagents is located closer to the periphery of the microarray than the analyte-related binding element for the same analyte, a device, wherein a part of the dissolvable elements are located in an area of the detection surface comprising the center of the microarray and a part of the dissolvable elements are located in an area of the microarray located relatively closer to its periphery, so that preferably, when the liquid sample is applied to the center a “later” assay component located relatively closer to the periphery of the microarray is dissolved differently time-wise to the “early” components that are located relatively closer to the centre of the microarray, preferably dissolvable elements are co-located, which provides a more even distribution of these dissolvable elements, wherein preferably the liquid sample is applied to the center of the detection surface.

In a particular embodiment of the method a device is used, wherein the periphery of the microarray is proximal a container side-wall and comprises only dissolvable elements so that it becomes free of said elements after the addition of the sample liquid on the detection surface so that there is a reduced risk that reflections from the side-walls would distort an optical reading, wherein preferably the liquid sample is applied to the detection surface so that it is evenly distributed thereon.

In a preferred embodiment of the method each of the one or more dissolvable target detection reagents is capable of binding an analyte (i.e. being an analyte detection reagent), each being adapted to bind a different analyte.

In an embodiment of the method the total volume of the liquid sample to be measured is added to the detection surface, e.g. wells, in steps consisting of small volumes thus controlling certain parameters that influence conditions for the reactions inside the wells, supporting the optimization of the reactions.

Usefully, bound elements are detected by an optical detection device, and the reference binding elements, when present, are located in the microarray to take into account the optical properties of the optical detection device thereby avoiding or reducing errors in a standardization and calibration signal.

### BRIEF DESCRIPTION OF THE DRAWINGS

FIGS. 1A, 1B, 1C, and 1D illustrate an example of the construction of different dots of a microarray of an embodiment according to the present invention;

FIG. 2 illustrates an embodiment of an arrangement of dots in a microarray according to the present invention;

FIG. 3 illustrates the arrangement of dots of FIG. 2 after the addition of liquid sample to the microarray and the dissolving of dissolvable dots;

FIG. 4 illustrates the chemical reaction resulting from the addition of a liquid sample to the microarray;

FIGS. 5A, 5B, and 5C are images showing the results of an experiment that exemplifies one of the embodiments of the invention;

FIGS. 6A and 6B illustrate an example of the construction of different dots of a microarray of a further embodiment according to the present invention a) before and b) after addition of a liquid sample;

FIG. 7 illustrates an example of the construction of different dots of a microarray of a further embodiment according to the present invention; and

FIG. 8 illustrates the arrangement of dots of FIG. 7 after the addition of liquid sample to the microarray and the dissolving of dissolvable dots.

### DETAILED DESCRIPTION OF THE INVENTION

As used herein:

The term “microarray” shall mean a two-dimensional array of small quantities of bio-chemical material, used for various types of assay. In this context, it is clear that the positions of the quantities may be selected freely, as a number of advantages are seen in different positioning schemes. The elements and positions of the detection surface may be positioned in a “microarray”.

The term “fluorogenic ligand” shall mean a ligand that is not itself fluorescent, but becomes fluorescent when it is bound by another substance, such as a molecule, chemical specific protein, RNA or DNA structure.

The term “replicate” shall mean a copy of a particular binding or dissolvable element at a discrete location in a microarray, but which may be present in different concentrations or in different locations in the microarray in different instances of the microarray or assay.

The term “liquid sample” shall mean any liquid comprising one or more analytes. Liquid samples may e.g. be plants or meat extracts, milk, serum, beverages or waste water.

The term “matrix effect” shall mean the effect of overall composition of a liquid sample on the chemical reactions in an assay and may be defined as a negative effect on the performance of the assay in a complex matrix in a sample such as from e.g. plants or meat extracts or milk or serum.

The term “analyte” shall mean a natural or synthetic substance or compound, preferably a molecule, or a biological cell, preferably a micro-organism, which is a component of a liquid sample which is to be detected by the device of the invention either quantitatively or qualitatively.

The term “analyte analogue” shall mean any constituent component of an assay that is not originally found in the liquid sample whose binding properties, in particular specific affinity and binding interactions are the same, complementary or similar to those of the analyte with the constituting components of the assay that are designed to bind the analyte (binding elements). Preferably the analyte analogue has the same binding properties as the analyte. Also preferably the analyte analogue has similar binding properties as the analyte to the extent that a binding element capable of binding the analyte is capable of binding the analyte analogue as well. Preferably the analyte analogue is a labelled or unlabelled ligand or receptor or nucleic acid polymer or any other entity,

The term “target” shall mean any component or element in an assay that is to be reacted with or detected or to which a binding element is designed to bind to.

Accordingly, “analyte” and “analyte analogue” are both a specific variants of a “target”.

The term “assay element”, or first/second component, shall mean any constituent component of an assay that is not introduced to the assay as part of the liquid sample. An assay element can be a part of the microarray, be added in a liquid or powder form to the liquid sample or present in or added to a buffer being applied as component of the assay.

The term “container” shall mean a hollow object, preferably a receptacle, for holding elements and/or reagents and is suitable for containing liquid, such as a liquid sample. Preferably the container according to the invention has a floor with walls to hold the liquid. Preferably the container according to the invention is round or has a circular cross-section. The container can be e.g. a vial or a well e.g. of a well-plate or a slide.

The term “detection surface” shall mean a physical part of the device of the invention to which elements and reagents are attached to and on a portion of which the signal is evolved allowing detection of molecular binding events; the detection surface can be the bottom of a container, e.g. a vial or a well e.g. of a well-plate or a surface of a slide. The detection surface may have a centre, which may be any position on the detection surface.

The term “soluble binding” shall mean a binding by which a reagent or element is bound to the detection surface, wherein said binding is ceased upon applying a liquid, e.g. a liquid sample to the surface thereby releasing said reagent or element into the liquid. Attachment is another term for the same functionality.

The term “insoluble binding” shall mean a binding which does not cease, i.e. remains intact, upon applying a liquid, e.g. a liquid sample to the surface; the binding may be a covalent binding or a strong secondary binding.

The term “immobilized binding element” shall mean an element which is not easily removed from the detection surface by any action of the liquid sample, such as by a flow thereof or by the liquid sample potentially dissolving the immobilized binding element. The immobilized binding elements may be attached to the detecting surface using insoluble binding. The immobilized binding element may be configured to remain in its position for at least a period of time during which the device is in use, such as from receiving the liquid sample and until a detection has been performed. The immobilized binding element preferably is configured to remain in place for at least twice that period of time.

The term “binding element” and “binding component” shall mean a substance which comprises a compound or molecule preferably a molecule capable of binding to a molecule of interest; preferably the binding element of the invention is immobilized to the detection surface.

The term “specific” shall mean a given entity, e.g. a given target. If binding of a given target is specific it is to be understood that said binding is stronger, preferably significantly stronger to that specific target than to other targets in the assay or on the assay surface or in the liquid sample.

The term “analyte-related binding element”, “analyte-related binding component” or “binding component”, shall mean a binding element which is capable of binding a compound or molecule which is related to an analyte i.e. either is an analyte-analogue or comprising an analyte-analogue or is a compound capable of binding an analyte, whereby binding results in a complex which comprises an analyte and which is suitable to obtain a signal which indicates that said binding occurred. Said signal may be obtained either in the same binding step or in a further step or steps in which, however the complex is maintained. In an embodiment the analyte related binding element comprises an analyte-analogue according to the invention and is capable of binding a soluble compound or molecule capable of binding the target. In other embodiments the analyte related binding element is capable of binding an analyte. Preferably the analyte-related binding element is specific to a given analyte, e.g. comprises a given analyte or is capable of binding a given analyte specifically. The specific analyte-related binding elements may be specific analyte binding elements, e.g. “capture spots” or “capture dots” if provided on the detection surface in a spot or dot form.

The term “dissolvable element” shall mean a substance which comprises a compound or molecule which is bound by soluble binding to the detection surface. Preferably, the soluble binding is configured to allow that at least 10%, such as at least 25%, such as at least 50%, such as at least 75%, such as at least 90% of the dissolvable element is dissolved within no more than 90% of the above period of time, such as within no longer than 75% of the above period of time.

A position of an element may e.g. be any position within a contour of the element projected on to the detecting surface. The position may be a centre of a contour of the element projected on to a plane of the detecting surface. The centre may be a geometrical centre.

The term “target detection reagent” shall mean a compound or molecule which is capable of binding to a target, e.g. an analyte or an analyte-analogue and thereby results in or takes part in or mediates the obtaining of a signal reporting on target presence.

A “specific target detection reagent” shall mean a compound or molecule which is specific to a given target, i.e. the binding of said reagent to a specific target related binding element results in a complex specific to the given target and the signal obtained either in the same binding step or in further step or steps will be a target-specific signal. A specific target detection reagent can be e.g. a labelled target species binding molecule or element or a labelled secondary specific target related binding element.

The term “reference binding element” and “reference binding component” shall mean a binding element which is capable of binding a reference reagent, preferably a dissolvable reference reagent, i.e. a reference compound or molecule which is non-related to any analyte i.e. is neither an analyte nor analyte-analogue nor comprises an analyte nor analyte-analogue nor is a compound capable of binding an analyte nor analyte-analogue but wherein said binding of a reference reagent occurs under conditions wherein an analyte related binding may occur. A reference binding element can be e.g. a labelled reference species binding molecule. The reference binding elements may be e.g. “reference dots” if provided on the detection surface in a dot form.

A reagent or element is “located” in a given position on the detection surface which is registered and which can be identified independently whether a signal is evolved or not on that location upon performing an assay with the device of the invention.

“Applying” a liquid, e.g. a liquid medium (like solvent or buffer or washing medium) or a liquid sample on the detection surface means any method or process resulting in a distribution of the liquid on the detection surface, preferably an even distribution once equilibrium is reached. Applying the liquid may be carried out by dispensing, e.g. by pipetting or dropping or pouring or soaking, preferably pipetting. In an embodiment applying may include sedimentation e.g. centrifugation.

“Washing” the detection surface means herein the application of a liquid, preferably a liquid medium with the view of removing it afterwards and thereby removing any compound dissolved therein.

The term “tag” shall mean a moiety, i.e. a part of a molecule preferably a polypeptide molecule which has been artificially added, preferably a peptide sequence genetically grafted onto a recombinant protein and which allows specific binding of the molecule. For example such tag can be a His-tag or a FLAG-tag (FLAG is a common name for a specific short, hydrophilic, 8-amino acid peptide tag (DYKDDDDK tag) and evolved from a trade mark Flag™ that was introduced in 1988.)

The term “comprises” or “comprising” or “including” are to be construed here as having a non-exhaustive meaning and allow the addition or involvement of further features or method steps or components to anything which comprises the listed features or method steps or components.

The expression “consisting essentially of” or “comprising substantially” is to be understood as consisting of mandatory features or method steps or components listed in a list e.g. in a claim whereas allowing to contain additionally other features or method steps or components which do not materially affect the essential characteristics of the use, method, composition or other subject matter. It is to be understood that “comprises” or “comprising” or “including” can be replaced herein by “comprising substantially” if so required without addition of new matter. Unless otherwise defined, all technical and scientific terms used herein have the same meaning as commonly understood by one of ordinary skill in the art to which this invention belongs.

As used herein the singular forms “a”, “an”, and “the” each includes plural form as well unless the context dictates otherwise.

We include in our description some novel considerations and descriptions with regards to the spatial arrangement of the elements (a plurality of immobilized binding elements and a plurality of dissolvable elements) in a microarray and repetitions of some of them, proposing the reduction of distorting effects that may rise from typical chemical, mechanical, flow, and optical variations in the realization of the assay, as well as the detection of the measureable signals that it produces. For that purpose, in an assay device having a detection surface which is contacted with a liquid sample during operation of the assay, and which comprises a microarray of elements (typically and preferably dots applied previously on the detection surface), the dissolvable elements are preferentially located at the peripheral parts of the detection surface and/or the central part thereof, so that when the liquid sample is applied (e.g. dispensed) onto the surface, the dissolvable elements are removed from the detection surface into the liquid, i.e. dissolved into the liquid, leaving a surface area including the centre of the detection surface and/or a surface area proximal to the periphery of the detection surface, respectively, free of dissolvable elements. Exactly these parts of the detection surface are where detection is problematic e.g. because of uneven distribution of the liquid sample (e.g. it is typically applied on the central part of the assay) and/or because there are distorting effects either fluidically or optically, which is typical at the peripheral part of the detection surface.

A sample that can be used may be anything in food, feed, waste water, recycled waste water, agriculture and the like that can be dissolved or immersed into liquid. E.g., food and feed matrices as grains, cereals, pseudocereals (such as e.g. amaranth and quinoa), rice, raw milk, plant milk, dairy products, meat, plant based meat substitutions, seafood (fish and shellfish), seaweed, protein crops (such as e.g. soybean, peanut, common bean, pea, lupine, chickpea, faba bean, lentil, grass pea, cowpea, flax seeds, caraway seeds and pigeon pea), fruits, vegetables, eggs, nuts, species, herps, sugar, hemp, tobacco, beverages, cocoa beans, coffee beans, tea leaves, wine, beer, olive oil, oily seeds, starch, fat, dietary fibers, forage, etc., chemicals, drugs, toxins, preservatives, food additives, metals, ions, essential minerals, minerals, salts, vitamins, micro-organisms, viruses, nucleic acids, proteins, fungi, parasites, insects, animal tissue, and specialized types of cells such as e.g. somatic cells.

There are several embodiments of the assay of the invention a number of which is exemplified hereinbelow.

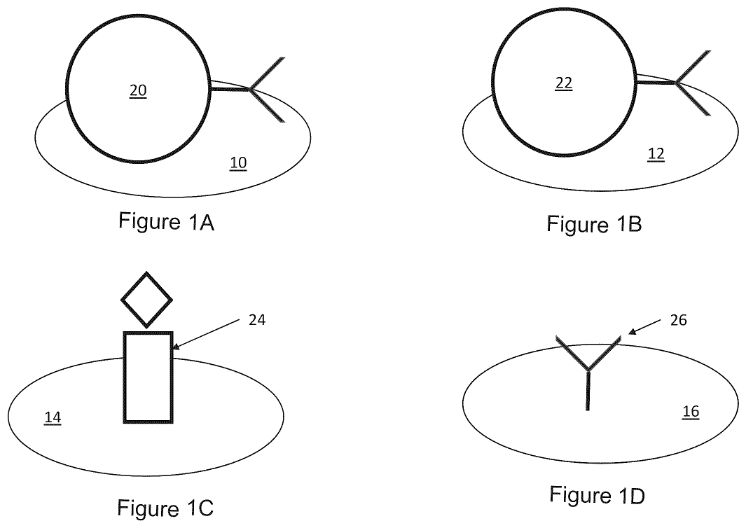

Considering now FIGS. 1A, 1B, 1C, and 1D, illustrating a dissolvable specific Mycotoxin marker dot 10 with an Anti-Mycotoxin antibody label 20, a dissolvable reference marker dot 12 with a non-specific antibody label 22, a specific Mycotoxin capture dot 14 with a Mycotoxin conjugate 24 and a reference dot 16 with an anti-species antibody 26, FIG. 1A illustrates an exemplary dissolvable target detection reagent consisting of a specific mycotoxin marker, that is immobilized at a location as dots in a microarray on a detection surface by a weak (“soluble”) bond, which allows it to dissolve into sample liquid, once the liquid is added. The mycotoxin marker comprises a label molecule conjugated to a specific mycotoxin antibody (anti-AFT, anti-DON, anti-FUM, anti-OTA, anti-T2, or anti-ZEA). A plurality of dissolvable target detection reagents having different specific mycotoxin markers are required in a multiplex mycotoxin assay to detect a plurality of different mycotoxin analytes in the liquid sample. An exemplary dissolvable reference reagent or “reference marker” dot 12 is illustrated in FIG. 1B and consists of a label molecule (circle) that is bound to a non-specific (reference) antibody or a protein (Y-shape), such as any antibody that doesn't have any affinity to the analyte mycotoxins of interest, that is immobilized at the bottom of the dot by a weak bond. This allows the reference marker dot to dissolve into the liquid sample, once it is introduced to the microarray. An example of an analyte-related binding element or “capture dot” 14 is illustrated in FIG. 1C. The capture dot of this embodiment consists of an analyte analogue, which is a specific mycotoxin conjugate between an insoluble agent that fixes the conjugate to a discrete location in the microarray on a detection surface, e.g., a protein and a specific mycotoxin (Aflatoxin “AFT”, Deoxynivalenol abbreviated “DON”, total Fumonisin “FUM”, Zearalenone “ZEA”, T-2, and Ochratoxin A “OTA”) that has specific affinity to the respective specific mycotoxin marker. FIG. 1D illustrates a reference binding element or a “reference capture dot” 16 consisting of a reference binding element comprising an anti-species antibody 26 that binds to the non-specific antibody of the reference marker. For ease of visualization and of understanding each dot of FIGS. 1A to 1D is shown to consist of only one element but it will be understood that each dot may comprise a plurality of elements without departing from the invention as claimed.

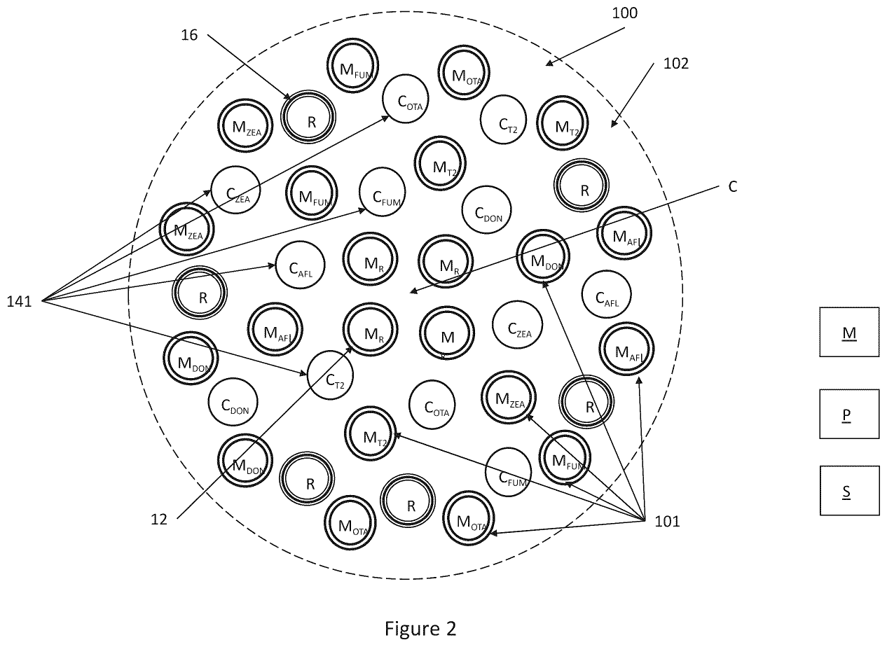

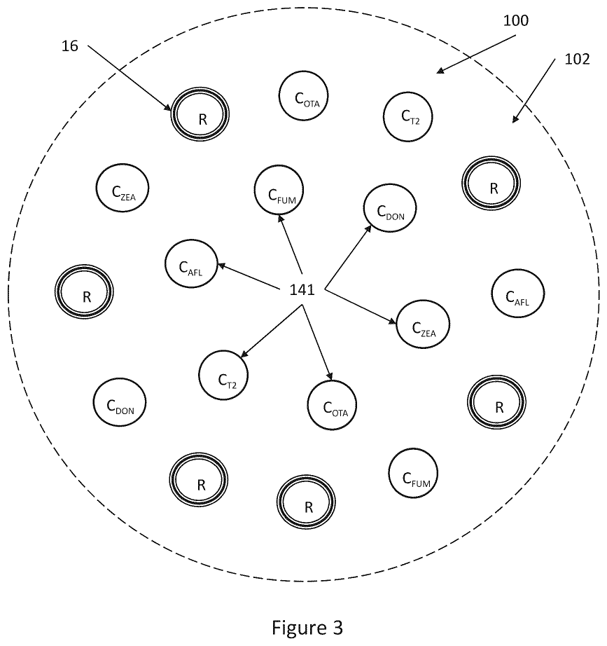

One embodiment of a system for the determination of one or more of multiple analytes in a liquid sample according to the present invention is illustrated in FIG. 2 and FIG. 3. As illustrated in FIG. 2, the system comprises a circular microarray 100 of immobilized binding elements consisting of a plurality of different specific analyte-related binding element replicates 141, such as Aflatoxin capture dots, (labelled “Cₓₓₓ”—where the index XXX indicates the specific mycotoxins AFT, DON, FUM, ZEA, T2 and OTA described above) and a replicate reference dot or binding element 16 (labelled “R”), and of dissolvable elements consisting of a replicate dissolvable target detection reagents 101, such as an aflatoxin marker, (labelled “Mₓₓₓ”—where the index XXX corresponds to the labels associated with the analyte-related binding elements) and a replicate dissolvable reference reagent or marker 12 (labelled “M_{R}”). The immobilized binding elements are immobilized by insoluble binding at discrete locations on a detection surface 102, for example a floor of a circular well as illustrated by the broken circle in the figures, and the dissolvable elements are immobilized by soluble binding at discrete locations on the detection surface. As illustrated in FIG. 2, a first group of the dissolvable elements are located closer to the center, C, of the microarray and a second group is positioned closer to the periphery of the microarray than the immobilized binding elements. As is illustrated in FIG. 3, once liquid sample, typically containing analytes to be assayed, is introduced to the microarray the dissolvable elements move from the detection surface and into the liquid, leaving the centre and the periphery of the microarray free of elements.

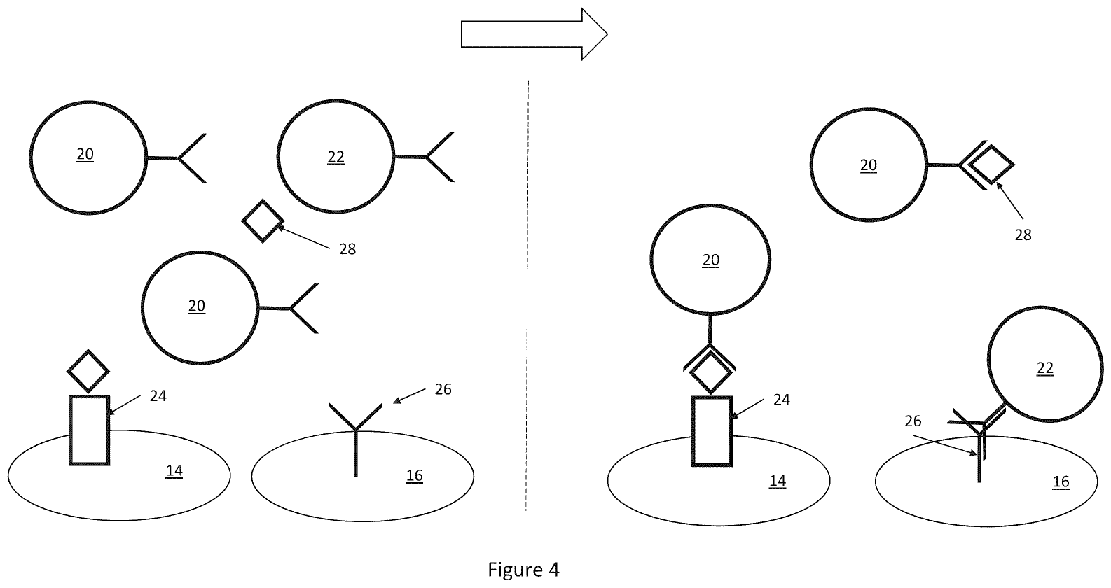

The reactions which take place upon the introduction of a liquid sample containing analytes to be assayed are illustrated in FIG. 4 by way of example. The reagents at the marker dots, that is the different specific mycotoxin (or analyte) dissolvable target detection reagents and the non-specific dissolvable reference reagent, are dissolved into the liquid sample. Competitive binding reactions (arrow) then take place, in which the mycotoxin-conjugates in the different analyte-related binding element replicates compete with the associated sample mycotoxins 28 (analytes) present in the liquid sample for binding to the respective antibodies of the detection reagents (Y-shaped portion of the label 20). Non-specific (reference) (Y-shaped portion of the label 22) markers bind to the anti-species antibodies of the reference binding elements. After a fixed reaction time, of typically and non-limitingly around five minutes, the liquid is removed from the microarray, preferably but not essentially washing is performed, typically and non-limitingly for around one minute, for example using a phosphate buffered saline (PBS) solution, taking with it the unbound detection reagents, sample mycotoxins and the excess reference reagent that remained unbound in the liquid. The concentration of the target detection reagent that remains bound to the analyte-related binding elements of the capture dots will be inversely-proportional to the concentration of the mycotoxins present in the sample. The concentration of the reference reagent that remain bound to the reference binding elements of the reference dots, indicate the conditions under which the chemical binding reactions took place, and are used for standardizing the readings from the analyte related binding elements.

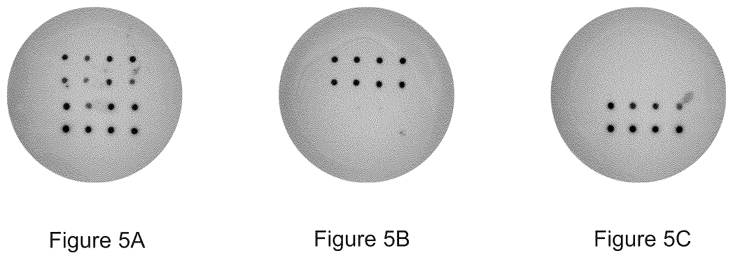

The results of assays performed using a further embodiment of a system according to the present invention in which the microarray is realized as a rectangular array are illustrated in the FIGS. 5A, 5B, and 5C. Anti-FUM- (i) and anti-DON-antibodies (ii) were spotted by insoluble binding in a 4×4 arrays of analyte-related binding elements in rows 1-2 (i) and rows 3-4 (ii), respectively. The rows are numbered from top to bottom. Dissolvable DON-HRP and dissolvable FUM-HRP analyte related target detection reagents were spotted closer to the well walls, peripheral to the microarray, than the analyte-related binding elements, but these dots of dissolvable target detection reagents are not coloured and cannot be observed in the images. FIG. 5A is the resulting image of the colour developed at analyte related binding elements of the microarray by the reaction of horseradish peroxidase (HRP) and precipitating 3,3′,5,5′-Tetramethylbenzidine (TMB), in the case where the microarray is exposed to a liquid sample containing no DON, nor FUM. FIG. 5B is the resulting image of the colour developed at the analyte-related binding elements of the microarray by the reaction of HRP and precipitating TMB, in the case where the microarray is exposed to a liquid sample containing high concentration of DON, but no FUM. It can be seen that the high concentration of DON prevents binding of DON-HRP to the dots of rows 3 and 4 and therefore no signal is developed. FIG. 5C is the resulting image of the colour developed at the analyte-related binding elements of the microarray by the reaction of HRP and precipitating TMB, in the case where the microarray is exposed to a sample liquid containing high concentration of FUM, but no DON. It can be seen that the high concentration of FUM prevents binding of FUM-HRP to the dots in rows 1 and 2 and therefore no signal is developed.

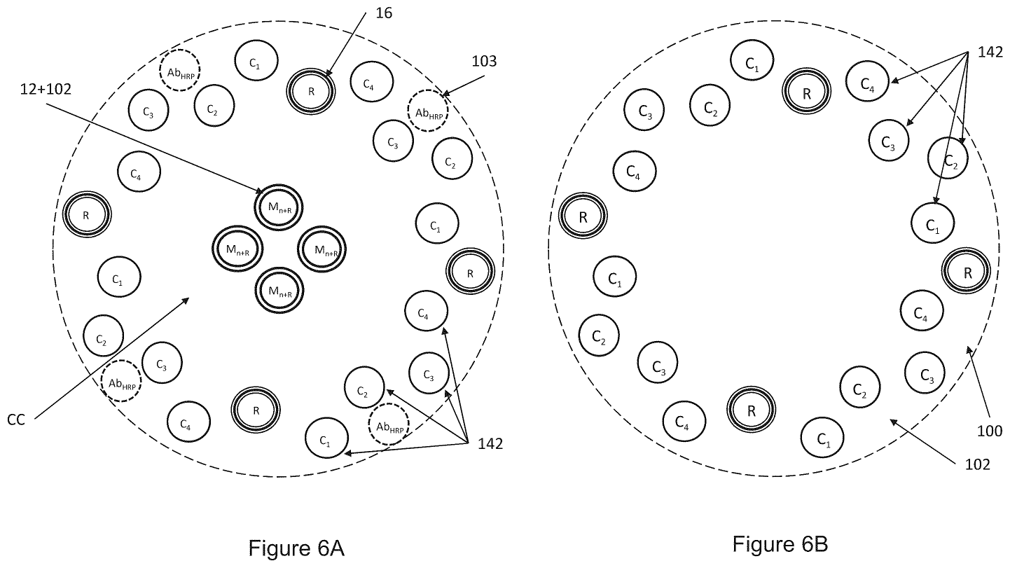

A further embodiment of a system according to the present invention is illustrated in FIGS. 6A and 6B. Here the subscript numbers 1 to 4 represent the antibiotics analytes Beta-lactams, tetracyclines, and sulphonamides, and the mycotoxin analyte aflatoxin M1, respectively. The respective analyte-related binding elements, also called “capture dots” 142, are, in this example, denoted C₁, C₂, C₃, C₄ and have a construction similar to the one described in the example of FIGS. 1A to 1D, which is based on analyte analogues, but of the analytes specified here and not the ones described in the example of FIGS. 1A to 1D. The dissolvable target detection reagents 102 are denoted Mₙ where the index n goes from 1 to 4 and denotes, as above, beta-lactams, tetracyclines, sulphonamides and the aflatoxin M1, respectively, and consist of binding agents, e.g. antibodies, to bind the respective analytes, or their analogues, which can optionally be conjugated to additional molecules, e.g. phycoerythrin, that can be used for labelling as will be described below. The dissolvable reference detection reagents 12, M_{R} comprises non-specific antibody or protein that bind to the antibodies that constitute the reference binding elements (“reference capture dots”) that are denoted by the index R. Accordingly M_{n+R} (12+102; FIG. 6A) denotes that in the present embodiment the dissolvable elements (Mₙ and M_{R}) are immobilized by soluble binding at the same discrete locations and are co-located closer to the center (preferably between the 12/102 dots) of the microarray than any of the insolubly bound immobilized binding elements. Co-locating the dissolvable elements makes the assay easier to produce and will generate an even more symmetric distribution of the markers than having individual dissolvable dots. Also included in this exemplary microarray of the present invention are HRP (horseradish peroxidase) labelled detection antibody dots 103 (denoted by Ab_{HRP}), which bind to the dissolvable target detection reagents. The HRP might of course be attached directly as a label to the markers/reference(s). However, here we illustrate that it may be of advantage in some cases to use the possibility to control that a “later” assay component located relatively closer to the periphery of the microarray is dissolved at a later time compared to the “early” components that are located relatively closer to the centre of the microarray. The HRP here has the same role as described in relation to FIGS. 5A to 5C, that of a catalyst which converts its substrate by an enzymatic reaction such that the chromogenic reagent precipitates locally and can be read as a stained dot. An alternative centre is illustrated at CC. Dispensing the sample at this position would have another effect on the flow and contents of the fluid and the analysis. Naturally, the 12/102 dots may be displaced to be positioned around the CC position to again arrive at an analysis as described above.

After addition of the liquid sample to the microarray, see FIG. 6B, the dissolvable elements are removed into the sample liquid to leave the centre of the microarray unpopulated by any element of the system. By immobilizing elements at the same location in this manner it is more simple to produce the dots of the microarray and is less complex to prepare the production of a well-plate containing the microarray of the present invention. Accordingly, the production of the microarrays is made faster, yielding shorter time from starting the production of the assays until moving the ready-to-use assays into optimal storage conditions, thus increasing the stability of the assays over time and reducing waste. The number of dots may vary from 1 of each to replicates of a high number.

In an embodiment of the invention the system is realized in cylindrical plastic tubes or vessels with a flat bottom. The said tube may be, such as, without any limitation, a well within a standard microtiter wells plate, a stand-alone tube, or any other assembly of tubes or vessels.

In some embodiments of the invention the center of the microarray consists only of dissolvable dots, so that it becomes free of dots after the addition of the sample liquid, which makes it available for pipette approach, in the case of instrumental realization of the assay. Additional advantages gained by designing the positioning of the different dots with regards to the pipette approach can be considered with regards to the chemical reactions taking place. For example, in the case in which the dissolvable marker dots (the dissolvable elements) are placed right under the pipetting position, their reaction with targets within the sample will start prior to the reaction of the targets with the specific binding agents in the capturing dots (analyte-related binding elements) (see Examples 1 and 6) influencing the kinetics of the reaction towards a steady state or equilibrium.

In addition to the above, the location and spread of the dots within the microarray can also be used to optimize the extent to which a measuring would be representative of the true content of the added sample.

The number of the dots may vary from 1 of each type to a high number. Preferably a plurality of dots of each type is present on the detection surface (i.e. each type of target and reference). The same consideration made above is true and it will be appreciated that it does not have to be 4 copies of each component.

In an embodiment of the invention the detection surface is provided as a flat face or surface of the bottom of a well, the periphery of the microarray consists only of dissolvable dots (the dissolvable elements), so that this peripheral part of the surface becomes free of dots after the addition of the sample liquid into the well, which reduces the risk that reflections from the side-walls of the well would distort an optical reading of dots.

In some embodiments reference dots (the reference binding elements) according to the present invention may be positioned within the microarray with reference to the position of the capturing dots (analyte-related binding elements), in order to facilitate normalization and standardization also with respect to the location on the microarray, compensating for possible variations in illumination and detection homogeneity, as well as some local chemical variations or depletions.

The spatial arrangement of the dots and of the repetitions of some of them as provided in the descriptions, aspects and claims below, may reduce distorting effects that may rise from typical chemical, mechanical, flow, sample matrix and optical variations in the realization of the assay, as well as the detection of the measurable signals that it produces. For example, when the periphery of the microarray has only dissolvable dots so that it becomes free of dots after the addition of the sample liquid into a well, the floor of which acts as a detection surface, there is a reduced risk that reflections from the side-walls would distort the optical reading of dots.

Moreover, when the center of the microarray has only dissolvable dots, it becomes free of dots after the addition of the liquid sample, which makes it available for pipette approach, in the case of instrumental realization of the assay. Otherwise, the insoluble, fixed dots (the immobilized binding elements) face the risk of being damaged by directly applying or aspirating liquid by the pipette placed vertically above them.

Additional advantages gained by designing the positioning of the different dots with regards to the pipette approach can be considered with regards to the chemical reactions taking place during the assay. For example, in the case in which the dissolvable marker dots (the dissolvable elements) are placed right under the pipetting position or at least between the pipetting position and the capturing dots, their reaction with targets within the sample will start prior to the reaction of the targets with the binding agents in the capturing dots (e.g. the analyte-related binding elements) (see Examples 1 and 6) influencing the kinetics of the reaction towards steady state or equilibrium. This could be of importance especially for rapid assays with short reaction times, where high accuracy is desired but neither equilibrium nor steady states are reached.

The location and spread of the dots can also be used to optimize the extent to which a measuring would be representative of the true content of the added sample.

In the assay the sizes of the dots, their concentration, and drying times are used in order to optimize the reconstitution time of their active components upon the addition of the liquid sample.

In an embodiment the analyte related binding elements are immobilized to a surface without or with a specific orientation that is aided by, to give an example, coating the surface with an Fc-binding protein, e.g., protein A or protein G.

In another embodiment linkers can be used to immobilize the analyte-related binding elements either directly to the surface or indirectly through e.g. linker attachment to a protein or an alternative substance that is coated to the surface.

For example positioning of the analyte-related binding elements and the reference binding elements enables characterization and control over parameters that indicate how well the conditions for an appropriate performance of the assay are fulfilled, e.g., how well the liquid sample is mixed and evenly distributed across the detection surface

In a variant, two or more identical measurement areas provided for the determination of each analyte or for physical or chemical referencing within a segment or array in order to monitor the level variations in the measured values and determining the background signal between or adjacent to the measurement areas. This is an example or a variant of an embodiment when the two or more measurement areas are relative to each other.

The addition of magnetic particles, application of centrifuging, or other methods, which can physically push targets (e.g. the analytes) towards the detection surface, e.g. the bottoms of the wells, may increase the interaction with binding elements (e.g. the analyte related binding elements), and contribute to the increase of the speed of measurements, along with increasing sensitivity and efficiency of the assays.

Different dot sizes, component concentration and conditions can affect the kinetics of the assay, and at what point in time each element and component is “joining the reaction”, determining to a certain extent the order of reactions and therefore influencing the “direction” it goes. This contributes to assay optimization in view of sensitivity and time to results.

In the following, the invention will be illustrated by means of exemplary embodiments which, however, are not to be construed as limiting the invention.

### EXAMPLES

### Example 1: A System for the Simultaneous Detection of 6 Mycotoxins in a Liquid Extract from a Ground Sample of Grain

The mycotoxins to be detected in this example are total Aflatoxin (AFT), Deoxynivalenol (DON), total Fumonisin (FUM), Zearalenone (ZEA), T-2, and Ochratoxin A (OTA).

Extraction from a ground specimen of grain is done by immersing the ground grain in Ethanol 23%, mixing well by shaking, and filtering to obtain an extract liquid.

Three types of dots are spotted in a microarray patterned onto a tube's bottom, which acts as a detection surface by adequate commercially available equipment, for example, the microarray printer available from Arrayjet Ltd., Stobo House, Roslin, United Kingdom, (see FIG. 1)—capturing dots (“analyte-related binding elements”, FIG. 1c)), reference dots (“reference binding elements”, FIG. 1d)), and dissolvable marker dots (“dissolvable elements”), wherein dissolvable marker dots may comprise two types of dot, i.e. dissolvable specific marker dots (“target detection reagents”, FIG. 1a)) and dissolvable reference marker dots (“dissolvable reference reagents”, FIG. 1b)). Capturing (or shortly: “Capture”) dots and reference dots are immobilized binding elements which are immobilised at discrete locations in the microarray on the detection surface by insoluble binding thereto. For each mycotoxin (i.e. analyte or target) 5 dots are dedicated—2 specific capturing dots and 3 dissolvable specific mycotoxin marker dots. Six non-specific reference dots and four dissolvable non-specific reference marker dots are also included and spotted onto the bottom of the well. In total, 40 dots are spotted onto the bottom of the well (see FIG. 2). The capturing and the reference dots are distributed in a way that would provide appropriate sampling and detection (see FIG. 3), with the dissolvable marker dots in the central area of the circle and in the periphery, so that after introducing the liquid sample into the well and the dissolving of the marker dots, the center or central area of the well will be free of dots and available for pipette approach, in the case of instrumental realization of the assay, and no detected dots would lie too close to the walls of the well, in order to avoid optical reflections upon optically reading the signal intensity (for example luminescence) from the dots.

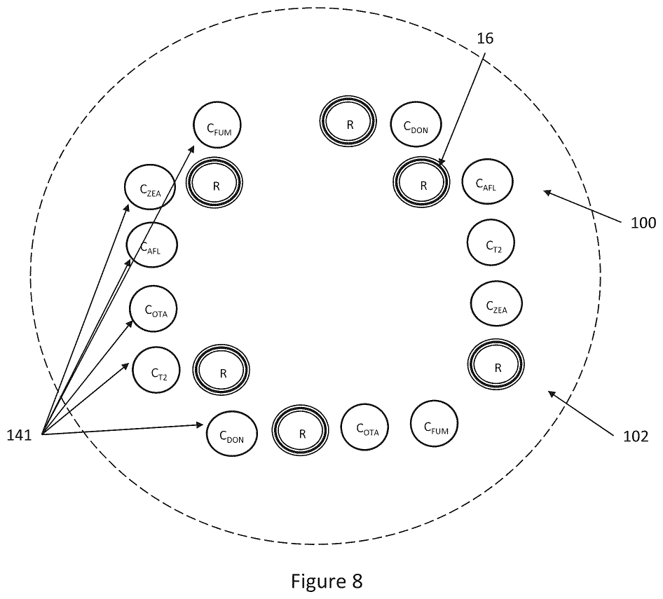

It will be appreciated that spotting geometries other than the circular geometry (see FIG. 2, FIG. 3 and FIGS. 6A and 6B) may be employed without departing from the invention as claimed, such as triangular, pentagonal, hexagonal, rectangular or the like geometry. A rectangular geometry is illustrated, by way of example only, in FIGS. 5A to 5C, FIG. 7 and FIG. 8. In this embodiment a rectangular microarray is provided which may, for example, be located on a detection surface on a well floor, such as a circular well floor, a rectangular well floor, or a well floor having other geometries, any of which may perhaps have rounded corners. The rectangular microarray here has replicates of dissolvable reference reagents (labelled “M_{R}”) and a plurality of different dissolvable specific target detection reagent replicates (labelled “M_{AFL}, M_{OTA}, M_{DON}, M_{ZEA}, M_{T2}, and M_{FUM}” respectively) immobilized by soluble binding at discrete locations, a portion of which are closer to the centre and another portion closer to the periphery of the microarray than are the insolubly immobilized replicates of the reference binding element (labelled “R”) and the replicates of the plurality of different analyte-related binding elements (labelled “C_{AFL}, C_{OTA}, C_{DON}, C_{ZEA}, C_{T2}, and C_{FUM}” respectively). The rectangular array has an advantage that it is, in general, more easily printed, such as by using commercial printer software and/or the commercially available printers for spot application.

The 6 different types of analyte-related binding elements, each corresponding to one of the mycotoxins to be detected, consist of a conjugate between a surface binding agent, e.g., BSA as carrier, and a hapten that is based on an analyte analogue to the mycotoxin to be detected.

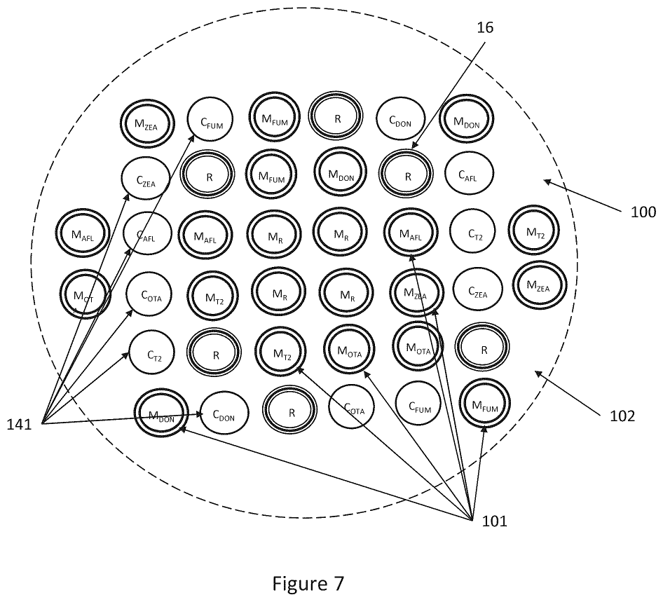

Six different types of dissolvable target detection reagent dots consist of a conjugation between a labelling agent, e.g., a gold nano-particle or a fluorescent molecule, and a mycotoxin specific antibody, one against each one of the mycotoxins (analytes) to be detected. In the accompanying figures they are called ‘mycotoxin markers’ (see FIG. 2 and FIG. 7). The other kind of markers are the reference markers (“dissolvable reference reagents”). These markers comprise the conjugations between a labelling agent (see FIGS. 1A to 1D and FIG. 4), e.g., gold nano-particles or fluorescent molecule, and a non-specific antibody (with no cross-reactivity with any of the anti-mycotoxin antibodies, or the mycotoxins themselves). It should be noted that the same labelling agent, e.g., gold nano-particles, can be used for all markers, because the different dots in the microarray (and thus the multiplexity of the assay) are identified and differentiated by their position in the microarray and not by the characteristics of the signal emitted from them.

The reference binding element dots consist of anti-species antibodies against the non-specific antibodies that are conjugated to the label molecules of the reference markers, having no affinity to the aforementioned specific antibodies or to any of the mycotoxins to be detected (analytes). These antibodies are immobilized to the surface of the bottom of the well (see FIGS. 1A to 1D and FIG. 4).

All dots are typically obtained by spotting sub-microliter volumes of liquid optimized buffers, containing the said reagents. The spotting is done by dedicated printers that create, control, and maintain the optimal condition for homogenous and reproducible spotting and drying of the dots. The size of the resulting dots of the microarray can range in scale from micrometers to millimeters. After spotting the capture and reference dots, a blocking or surface cover step might be needed before spotting the marker dots.

A fixed mark, e.g., a dot with a chromo- or fluorophore detectable by the detection unit, might be added in order to indicate orientation for the correct identification of the different dots upon analysis of the results, especially in the case of a stand-alone tube. In this specific example (FIGS. 2 and 3) there is no rotational symmetry in the distribution of the dots, and reference dots lie closer to each other, so the correct identification of the dots is straight forward.

Using the assays for the simultaneous measurement of mycotoxins consists of the following basic steps:

1.) Extracting the mycotoxins from a ground (e.g. grain) specimen into a liquid extract and typically filtering it and diluting it in order to reduce matrix and liquid extract effects.

2.) Dispensing a liquid sample of the diluted and filtered liquid extract in the well having the above-described printed dot micro-array at its bottom surface.

3.) Shaking the well or stirring the liquid extract so that the marker dots dissolve into the liquid and the immuno-reaction starts, consisting of (see FIG. 4):
Target mycotoxins (analytes) are bound by the respective specific markers in the liquid (e.g. AFT or AFL molecules are bound by the corresponding AFT-marker or AFL-marker molecules that contain corresponding anti-AFT or anti-AFL antibodies, etc.)
Unbound specific markers bind to the respective capturing dots.
Non-specific markers bind to reference dots.

4.) Emptying the well, and typically washing it with a wash buffer, in order to remove specific marker molecules that are bound by the mycotoxins (analytes) and the excess markers, if any, whereas the specific marker molecules bound to the capturing dots remain on the dots array.

5.) Illuminating and acquiring an image of the dots at the bottom of the well in the wavelength ranges that are optimal for obtaining optical signals from the different dots in the array with the best signal to noise ratio.

6.) Calculating the different mycotoxin concentrations on the basis of the intensity of the optical signals that come from the capture and the reference dots; e.g. on the basis of calibration curves that relate known concentrations of the analytes to be measured with the intensity of the optical signals that are recorded from the respective capturing and reference dots. These calibration curves may be produced in a known manner by performing a sufficient amount of measurements of samples with different, known concentrations of the measured analytes. The known concentrations are obtained typically by certified reference methods, e.g. high performance liquid chromatography (HPLC), but can also be obtained in some cases by spiking blank samples with known amounts of the target specimen. In these measurements the signals that are recorded from the reference are used for normalizing the signals that are recorded from the capturing dots. The normalizations can be done by different approaches and arithmetic operations, e.g., by dividing the signal recorded from capturing dots by the signals recorded from the respective reference dots.

Note that:
The higher the concentration of target mycotoxins in the extract is, the larger is the amount of respective specific marker molecule that gets bound by these mycotoxins, and which is removed by the washing step 4.), leaving a smaller amount of specific marker molecules that can bind to the capture dots resulting in a lower signal that is eventually obtained from the respective capturing dots.
The amount of non-specific markers that binds to the reference dots doesn't usually depend on the concentration of mycotoxins in the extract but on the environmental conditions that influence all the immuno-like chemical reactions that take place, e.g., temperature, pH, matrix factors, and more. These conditions will typically also affect the specific mycotoxin bindings, explaining how the reference dots serve for standardizing the results on the basis of reading the optical signals coming from the dots in the array.

It should be noted that the number of analytes that is addressed by this example was chosen only for the purpose of illustration only (six different mycotoxins) and can be enlarged significantly by e.g. adjusting the size of the dots and the concentration of the reagents in each one of them. Additionally, if needed, in an alternative embodiment of this example the specific mycotoxin marker dots (“specific target detection reagents”) can be removed from the microarray and the corresponding markers can be introduced to the assay as assay elements that are already dissolved into a liquid, which is added to the sample liquid, before the latter is dispensed onto the microarray. In such cases the said advantages obtained by the above described distribution of the dots in the microarray, so that the center and the periphery of the array remain free of dots after the addition of the sample liquid, is still maintained by placing the dissolvable reference marker dots (“dissolvable reference reagents”) in these regions.

In another additional embodiment of this example the specific mycotoxin markers can remain in dots form as originally illustrated in FIG. 2, but the reference marker (“dissolvable reference reagent”) be introduced to the assay as assay elements that are already dissolved into a liquid, which is added to the sample liquid, before the latter is dispensed onto the microarray. The surface area that will be freed in this case could be used to increase the number of different analytes addressed by the assay by adding more capturing dots and more dissolvable specific marker dots.

In yet additional embodiments of this example for one or more analytes the specific marker reagents can be introduced as dissolvable dots and for the other analytes as assay elements that are dissolved in a liquid that is added to the sample liquid, before the latter is dispensed onto the microarray. The choice of which markers should be introduced as dissolvable dots and which ones as assay elements that are dissolved in a liquid would typically depend on the relative affinities and the kinetics of the binding reactions for each one of the analytes, where the preference would be to add the marker in as assay elements in a liquid form in these cases where the marker affinity to the capturing dots would be relatively higher than its binding affinity to the analyte. In such cases adding the marker added as an assay element in a liquid form would give more time for the binding between the corresponding analytes and markers than in the cases in which the specific markers are introduced as dissolvable dots.

Moreover, any combination of the above embodiments could be considered and the assay elements that are added as a liquid, could be dispensed into the well or onto the detection surface before, together with, or after the liquid sample is dispensed into the well without altering the essence of this example and the fact that after adding the sample liquid into the well with the microarray at its bottom, the center and the periphery of the array will remain free of dots, as illustrated in FIG. 3. Furthermore, the choice of when to add the liquid containing the assay elements could be related to the kinetics of the different chemical reactions and the different binding affinities of the different reagents.

### Example 2: A Simultaneous Detection of Two Different Mycotoxins in a Liquid Extract from a Ground Sample of Grain

In this example the capturing dots (analyte-related binding elements) consist of anti-DON- and anti-FUM-antibodies, separately. The dissolvable specific marker dots (target detection reagents) consist of the antigens DON and FUM that are each, separately, conjugated with horse radish peroxidase (HRP) in order to form DON-HRP dissolvable dots and FUM-HRP dissolvable dots. A chemical blocking of the surface at the bottom of the well takes place after spotting the capturing dots. This is done by applying e.g. non-fat dry milk powder, in order to prevent unwanted and non-specific binding to the dots or to surface area around them.

Upon the addition of a liquid sample the DON-HRP and FUM-HRP conjugates, in the dissolvable dots, are dissolved into the liquid and compete with DON or FUM analyte molecules, which might be present in the liquid sample, for binding to the DON- and FUM-antibodies, respectively, in the capturing dots. The higher the concentration of the DON or FUM molecules in the liquid sample, the higher the number of DON- and FUM-antibodies in the capturing dots that bind to them, respectively, leaving a smaller number of antibodies in the capturing dots that are available to bind the DON-HRP and FUM-HRP, respectively.

The liquid is then washed away from the well and the number of DON-HRP and FUM-HRP that remain bound to the respective capturing dots is inversely-proportional to the concentrations of the DON and FUM molecules in the original sample.

Upon the addition of precipitating TMB (3,3′,5,5′-Tetramethylbenzidine) substrate (in this particular case SeramunBlau® spot by Seramun Diagnostica GmbH, Germany), a reaction that is catalyzed by the HRP that is bound to the dots produces a dark colour that is confined to the surface of the dots. The darkness of the dots is proportional to the concentration of the bound HRP at each dot, thus inversely proportional to the concentration of the DON and FUM in the liquid sample in the corresponding DON and FUM capturing dots.

The embodiment of Example 2 is illustrated in FIGS. 5A to 5C, in which images of 4×4 microarrays of capturing dots of anti-DON- and anti-FUM-antibodies are shown. Rows 1-2 consist of anti-FUM antibodies, and rows 3-4 consist of anti-DON antibodies, separately, which are spotted in at the bottom of a 96-wells plate. The rows are numbered from top to bottom in FIGS. 5A to 5C. These dissolvable DON-HRP dots and the dissolvable FUM-HRP were spotted closer to the well walls, peripheral to the microarray of the capturing dots, but these dots are not coloured and cannot be observed in the images. Upon the addition of blank samples, which neither contain DON nor FUM, the DON-HRP and FUM-HRP dots are dissolved into the liquid and the DON and FUM capturing dots are binding their maximal capacity of DON-HRP and FUM-HRP, respectively, as the absence of DON and FUM in the blank sample leaves free all the antibodies in the capturing dots for the HRP-conjugates binding. After washing the wells and adding the aforementioned precipitating TMB, all capturing dots are coloured to their maximum capacity, as can be seen in FIG. 5A. Alternatively, in cases that the liquid sample contains very high concentration of DON or of FUM, enough to bind the maximum number of anti-DON- or anti-FUM-antibodies in the capturing dots of rows 3-4 or rows 1-2, respectively, no antibodies remain available to bind DON-HRP FUM-HRP, respectively, that are dissolved by the sample liquid from the dissolvable dots. Accordingly, after the wash and the addition of precipitating TMB, in the case of a sample containing very-high concentration of DON and no FUM, rows 1-2 get strongly coloured, while rows 3-4 produce no colour (see FIG. 5B), and, in the case of a sample containing very-high concentration of FUM and no DON, rows 3-4 get strongly coloured, while rows 1-2 produce no colour (see FIG. 5C).

### Example 3: Detection of Different Specific Bacteria in Liquid Samples by a Protein Microarray and a Fluorescent Non-Specific Staining of the Nuclei of the Bacteria

In this example the analytes to be detected are specific bacteria in liquid samples, e.g., milk. The capturing dots (analyte related binding elements) comprise bacteria specific binding agents, typically antibodies, antibody fragments or recombinant proteins, that bind certain epitopes or ligands on the specific bacteria walls. The dissolvable marker dots (dissolvable target detection reagents) consist of a nucleic acid stain, e.g., Ethidium Bromide (EtBr), that binds non-specifically to the RNA and the DNA of bacteria. Thus, these are non-specific target detection reagents.

The simultaneous detection of different specific bacteria is achieved in a similar way as described in Example 2 above by spotting correspondingly different bacteria specific binding agents, as the capturing dots of the microarray.

After spotting the capturing dots, the bottom of the well is chemically blocked by, for example, skim milk, in order to prevent unwanted and non-specific binding to the dots or to surface area around them. The marker dots are spotted afterwards, including the aforementioned nucleic acid stain and additional reagents that aid with permeating the bacterial cell walls for the nucleic acid stain.

Upon the addition of the liquid sample to the well, the non-specific nucleic acid stain together with the cell permeating agents are dissolved into the liquid, staining the bacteria in the milk. In parallel, specific bacteria are bound (captured) to the capturing dots by the reaction between the specific binding agents in the dots and corresponding epitopes or ligands on the bacterial cell walls.

Different approaches can be applied in order to facilitate a good contact between the bacteria and the capturing dots at the bottom of the well, thus increasing the binding efficiency, the sensitivity, and the speed of the measurements. One of them can be the centrifugation of the well, or plate, in the case of well plates. Another one can be binding the specific bacteria (analytes) that are to be detected to magnetic micro- or nano-particles. This binding is enabled by coating the magnetic particles (functionalizing them) with binding agents that also bind to epitopes or ligands on the cells walls of the specific bacteria that are targeted. These binding agents can be the same as the ones constituting the capturing dots or alternatives that target the same specific bacteria, making this a sandwich assay. The functionalized magnetic particles are introduced to the liquid milk sample in the form of dissolvable dots similar to the dots described in FIG. 1A, as part of the microarray. They can alternatively be mixed with the liquid before it is introduced in the well. Once bound to the magnetic particles, the bacteria can be attracted to the bottom of the wells by introducing a magnet beneath it. The magnetic conjugates are attracted by application of a magnetic field to the detection surface that contains the insoluble capturing dots (analyte-related binding elements), pulling with them the analytes that are subsequently specifically captured by the said capturing dots. The magnet, or more generally the magnetic field, is removed afterwards and only the bacteria (analytes) that have been bound by the insoluble binding elements remain attached to the surface. The pulling and releasing, for example by bringing the magnet close and removing it, respectively or by alternately applying and removing a magnetic field, may be performed several times, perhaps with shaking in between, in order to increase the number of events in which the bacteria (analytes) meet their corresponding insoluble binding elements. Eventually the magnetic field is finally removed and only the bacteria (analytes) that have been bound by the insoluble binding elements remain attached to the detection surface. This process will typically end by aspiration of the detection surface, washing away all the unbound elements.

The use of magnetic particles in the above way shortens very much the time that it takes on average for an analyte or other target to come in contact with the binding agents in the capturing dots by putting on the targets a net force directed towards the detection surface. Without this force the targets reach the binding surface only due to sedimentation and diffusion, which are much slower processes. It is especially beneficial in cases where the targets don't bind easily to the binding elements on the detection surface, for example, in the case of bacteria. In some embodiments the magnetic particles are coated (functionalized) with reagents that universally bind non-specifically all bacteria, a large group of bacteria, or all bacteria of interest. In these embodiments all the bacteria in the assay will be attracted by the magnets to the detection surface, and as before only the bacteria that will meet binding elements that specifically bind them will remain captured at their corresponding dots and the specificity of the assay will be the same as the example above.

In some embodiments the magnetic particles are not detectable, and the assay also comprises additional dissolvable target detection reagents, that are immobilized separately. In other embodiments the magnetic particles are detectable (they can be e.g., fluorescent or chromogenic, along with being magnetic) and thus also serve as target detection reagents. In still other embodiments complexes may be provided in which the magnetic particles are both conjugated to analyte-related binding elements as well as to detectable (fluorescent or chromogenic) molecules to again serve as target detection reagents.

In certain realizations of this embodiment of the invention the nucleic acid staining together with appropriate cell permeating agents can be introduced in liquid form and mixed with the liquid sample, for example milk, before it is added to the well with the microarray at its bottom.

After enough time is given for the staining and the binding reactions to take place in the well, the liquid is washed out of the well, leaving inside it only the bacteria that have been captured by the capturing dots. As all the captured bacteria are also stained by the non-specific nucleic acid stain, e.g., EtBr, the fluorescence intensity that is measured from each capturing dot is proportional to the number of bacteria that have been captured by it. Thus, this is a direct non-competitive assay.

### Example 4: Detection of Different Specific Bacteria in Liquid Samples by a Protein Microarray and a Fluorescent Specific Cell Labelling of the Bacteria in a Sandwich Format

In this example the analytes to be detected are specific bacteria in, for example, milk samples. The capturing dots (analyte-related binding elements) consist of binding agents, typically antibodies, antibody fragments, or recombinant proteins that bind certain epitopes or ligands on the specific bacteria cell walls, similar to the case of Example 3 above. Different from Example 3 above, the dissolvable marker dots (dissolvable elements containing the target detection reagents) consist here of labelled binding agents that target epitopes or ligands on the bacterial cell walls. These can be the same epitopes or ligands that are targeted by the binding agents in the capturing dots or binding agents that target different epitopes or ligands than the epitopes or ligands that are targeted by the binding agents in the capturing dots.

Upon the addition of a liquid sample, for example milk, the labelled binding agents in the dissolvable dots, are dissolved into the liquid and bind corresponding specific bacteria in the sample. In parallel, same specific bacteria are binding (captured) to the capturing dots by the reaction between the specific binding agents in the dots and corresponding epitopes or ligands on the bacterial cells. Thus, this is a non-competitive sandwich assay. In order to facilitate a good contact between the bacteria and the capturing dots at the bottom of the well, thus increasing the binding efficiency, the sensitivity, and the speed of the measurements, centrifugation of the well, or plate, in the case of well plates, can be applied.

The higher the concentration of the specifically-targeted bacteria (analytes) the higher will be the number of bacteria captured by the respective capturing dots, and, as the captured bacteria will also be bound by the labelled-binding agents, the higher will be the intensity of the signal emitted from them. The intensity of these signals is recorded after washing the well and aspirating the liquid from it. Thus, this is a direct assay.

The agents that label the binding agents in the dissolvable marker dots (target detection reagents) can either be fluorescent molecules, e.g., Phycoerythrin or enzymes that catalyze a reaction that result in the development of colour upon the addition of TMB, e.g., HRP (see Example 2 above).

### Example 5: Detection of Different Specific Bacteria in Liquid Samples by a Protein Microarray and a Chromogenic Non-Specific Cell Labelling of the Bacteria by Nano-Particles

In this example the analytes to be detected are specific bacteria in e.g. milk samples. The capturing dots (analyte-related binding elements) consist of binding agents, typically antibodies, antibody fragments or recombinant proteins, that bind certain epitopes or ligands on the specific bacteria walls, similar to the case of Examples 3 and 4 above. Different from Examples 3 and 4 above, here the dissolvable marker dots (dissolvable elements) consist of binding agents that are labelled by nano-particles that produce colour when illuminated, of strengths that is proportional with their concentration. The labelled binding agents in the marker dots also bind epitopes or ligands on the bacterial cell walls. These can be the same epitopes or ligands that are targeted by the binding agents of the capturing dots or different ones.

Upon the addition of a liquid sample, for example milk, the nano-particles-labelled binding agents in the dissolvable dots, are dissolved into the liquid and bind corresponding specific bacteria in the sample. In parallel, same specific bacteria are binding (captured) to the capturing dots by the reaction between the specific binding agents in the dots and corresponding epitopes or ligands on the bacterial cell walls. Thus, this is a non-competitive sandwich assay.

In order to facilitate a good contact between the bacteria and the capturing dots at the bottom of the well, thus increasing the binding efficiency, the sensitivity, and the speed of the measurements, centrifugation of the well, or plate, in the case of well plates, can be applied.

The higher the concentration of the specifically-targeted bacteria (analytes) the higher will be the number of bacteria captured by the respective capturing dots, and, as the captured bacteria will also be bound by the labelled-binding agents, the higher will be the intensity of the analyte-related signals emitted from them. The intensities of these signals are measured after washing the well and aspirating the liquid from it.

In the specific examples given above and more generally using the system according to the present invention an automated pipette may dispense the sample in the center, in which case an advantage is reached in that the labelled elements are mixed with the analytes to be detected prior to the analyte related binding elements attached to the nano-particles. The binding kinetics may be very fast in the initial binding and can be influenced by giving the ligand or its competitive labelled partner a binding advantage. When doing short incubation assays to obtain fast answers, that do not reach binding saturation, this can have a significant on impact the test results.

The location and spread of the dots, in the examples above and the copies of each analyte-related binding element (e.g. Cn in FIGS. 2, 3, 6A, 6B, 7, and 8), as well as of the reference elements (e.g. R in FIGS. 2, 3, 6A, 6B, 7, and 8) secure that the measuring is more representative of the true content in the added sample.

### Example 6: A Microarray for a Simultaneous Testing of Antibiotics and a Mycotoxin in Liquid Samples

In this example the analytes to be detected are antibiotics and a mycotoxin in liquid samples, e.g. milk. The capturing dots (analyte-related binding elements) consist of binding agents, typically antibodies or receptor proteins, that bind the analytes to be detected, which in this example are specific antibiotics families, e.g., beta-lactams, sulphonamides, and tetracyclines, and mycotoxins, e.g., Aflatoxin M1. The specific dissolvable marker dots (target detection reagents) consist of antibiotics family members, e.g., Penicillin G, Sulfamethazine, Tetracycline, Aflatoxin M1, and the non-specific dissolvable marker, each conjugated with a label, e.g., Phycoerythrin. Furthermore, dissolvable detection dots consist of an enzyme, e.g. HRP, that is conjugated to an antibody which binds the label, e.g. Phycoerythrin.

This example is illustrated in FIGS. 6A and 6B that depict a microarray (FIG. 6A) in which the capturing dots (analyte-related binding elements) were spotted and dried in wells of a titer plate. The index 1-4 reflects beta-lactams, tetracyclines, sulphonamides and the mycotoxin aflatoxins M1 binding agents and the R reflects capturing dots of reference binding agent(s), respectively. Then, the wells were chemically blocked, washed and dried. Finally, the combined dissolvable marker dots and the reference dots (M_{n+R}) were spotted at the bottom of the same wells together with the dissolvable target detection dots (Ab_{HRP}) and dried.

In order to perform a measurement, a 150 μL milk sample is added and incubated while shaking at 37° C. for 5 or 10 min. The specific markers are dissolved into the liquid sample and compete with the corresponding members of the antibiotics families and the aflatoxin M1 potentially present in the sample on binding the binding agents in the capturing dots, which remain at the bottom of the wells as depicted by FIG. 6B. After a satisfactory reaction time, the liquid in the wells is aspirated and the wells are washed three times, leaving at the bottom of the wells HRP conjugated anti-Phycoerythrin antibody bound to Phycoerythrin labelled analyte analogues and reference marker(s). The binding to the capturing dots of analyte analogues occurs in competition with analytes in the sample. Thus, the concentration of the bound HRP at the capturing dots is inversely proportional to the concentration of the corresponding analytes in the liquid sample.

After the washing a precipitating TMB (3,3′,5,5′-Tetramethylbenzidine) substrate (in this particular case SeramunBlau® spot by Seramun Diagnostica GmbH, Germany) is added to the wells and starts a reaction that is catalyzed by the HRP that is bound to the capturing dots, producing a dark colour that is confined to the surface of the dots. The darkness of the dots is proportional to the concentration of the bound HRP at each dot, thus inversely proportional to the concentration of the members of the antibiotics families to be detected and the Aflatoxin M1 in the milk.

In this example, the dots can be measured with a known reader which is suitable for chromogenic detection. Additionally, it should be noted that the dots are not drawn proportional to the real size and may be varied in size and in distance. Smaller dots may be dissolved faster than bigger dots. The used concentration of the dot components may affect both dissolving speed and binding kinetics. The marker dots, for example, might be larger and vice versa. In this example one reference element and marker are used, but more than one may be used. The HRP conjugated antibody assay element may be part of the specific reference labelled elements and/or the specific marker elements or may be located proximate their location. The HRP conjugated antibody assay element may, in some embodiments, be added to the microarray by e.g. applying a buffer containing the antibody to the microarray. For the one known in the art a great variation of the above can be performed, for example the HRP labelled antibody and chromogenic steps may be omitted from the assay and the dots staining intensity from the bound marker and reference molecules may be read by e.g. a fluorescence reader.

An automated pipette may dispense the sample in the center, in which case an advantage is reached in that the target detection reagents are mixed with the sample prior to the analyte related binding elements in the capturing dots. Furthermore, a delay is also obtained before the liquid is reaching the non-specific marker dots, containing the (HRP-) enzyme-conjugated antibody, which binds to the target detection reagents. A further delay may be introduced by applying a sub portion of the sample or an equivalent volume of buffer covering only the central dots, then incubate shortly (e.g. a few seconds), followed by applying the sample rest volume. If the pipetting occurs above the dissolvable dots, then the risk of detaching a capturing dot caused by the speed of the dispensed liquid is reduced see, for example, FIGS. 6A and 6B.

In the case of, for example, very low concentration of analytes it might be advantageous to switch location between the specific and reference labelled elements and the specific binding elements in the examples illustrated in FIG. 6. This will provide a binding advantage for the analyte in the initial binding to the specific binding elements.

The dot's location in the microarray will for instance in case of a titer well geometry secure that the dot will obtain a more equal mix of the sample liquid during shaking, because of the more even distribution, in respect to the well geometry, compared to the geometries of traditional microarrays. The location of dots increases the chance of detecting errors occurring during the run e.g. air bubbles, shaker not working correct, non-homogenous sample etc. The fact that, for example, four dots are placed in a geometrically identical manner in a well means that they are all exposed to same conditions when the well/plate is shaken. This means that the variation among them will be decreased and a more representative result can be obtained compared with prior art. Furthermore, the amounts of replicates needed might be reduced and still obtain a better performance than traditional microarray assays, or, alternatively, the same amount of replicates can be kept and whilst securing a better performance than in prior art.

Such considerations and the organization of the dots can be used to optimize short incubation assays, because as mentioned in previous examples, and as the binding kinetic is very fast in the initial binding, the behaviour of the assay can be influenced by giving the detected targets or their competing specific markers an ‘advantage’ by letting them bind first.

The location and spread of the dots, as well as the number of replicates of each dot has, for example, the four replicates of each Cₙ and R in this example (FIG. 6), in the microarray secure that the measuring is more representative of the true content of the added sample.

The examples described hereinabove demonstrates that both competitive, non-competitive, sandwich, direct and even indirect assays will benefit from the invention. Moreover, not only microarray-based immunoassays will benefit from the invention, but assays for e.g. RNA and DNA detection may also benefit. This invention potentially improves the performances, by inventive dot distribution/geometric location and other suggestions for, for example, competitive, non-competitive, sandwich, direct and indirect liked assays for all types of microarray assays commonly known.

It will be appreciated that the invention as disclosed covers not only protein based microarrays but also microarrays based partially or fully on nucleic acids (DNA and RNA), and homologs thereof such as locked nucleic acids (LNAs), peptide nucleic acids (PNAs) and other synthetic nucleic analogs, or any modified forms of nucleotide polymers, that are capable of hybridizing with a target nucleic sequence by complementary base-pairing. Such assays can be used for detecting the presence of viruses or of organisms such as, but not limited to, prokaryotic or eukaryotic single, or multicellular cells, in a sample. The invention may also be employed for, for example, diagnostic purposes, such as detecting mutations and gene aberrations and for identifying genetically modified (GMO) food, crops or products. The target can be nucleic polymers from virus, human, animal, a specific cell type, plant, fungus, protist, bacterium, archaeon or artificial created. The sample may consist of any biological substance or purified fraction of such a substance. The nucleic based microarray shares the principles of the shown examples of how an assay can benefit of spot location and other advantageous considerations discernible from a consideration of the above. It is clear to a person skilled in the art that a hybridization microarray can be constructed in multiple ways whilst remaining within the scope of the present invention and thereby improve the overall assay performance compared to prior art.

### INDUSTRIAL APPLICABILITY

As described above, the present invention provides a device, a system and a method for the determination of one or more of multiple analytes as targets in a sample in such a way that the microarray on which the device, the system and the method are based, comprises of a plurality of binding elements immobilized by insoluble binding at discrete locations on the detection surface and a plurality of dissolvable elements immobilized by soluble binding at discrete locations on the detection surface. By introducing dissolvable elements the need for the manual addition of assay specific reagents is minimized or removed. By separating the dissolvable elements from the binding elements in a geometric advantageous manner provides an improved control of, amongst other things, kinetics influenced assay performance. By using geometric considerations in the location of the binding elements and the dissolvable elements, the microarray can be designed such that the replicates of each element have close to identical physical exposures to e.g. the sample, assay reagents and other assay liquids, for example, wash buffer, during liquid application, incubation and, when applicable, washing. By using said geometric considerations, the spatial arrangement of the binding elements and the dissolvable elements reduce distorting effect that may rise from typical chemical, mechanical, flow, sample matrix and optical variations in the realization of the assay, as well as the detection of the measurable signals that it produces.

### Aspects

1. A device for use in the determination of one or more of multiple analytes in a liquid sample, said device including a detection surface for exposure to the liquid sample and having thereon a number (microarray) of elements comprising:
a plurality of dissolvable elements which are dissolvable upon applying a liquid to the detection surface, said dissolvable elements being immobilized by soluble binding at discrete locations (in the microarray) on the detection surface, and
a plurality of immobilized binding elements immobilized by insoluble binding at discrete locations (in the microarray) on the detection surface, said binding elements include one or more of different analyte-related binding elements for each analyte, any or each of said analyte-related binding elements being adapted to bind to either or both an assay element and an analyte (to an extent that is related to the concentration of the analyte in the liquid sample), and which when bound, produce a detectable analyte-related signal for use in making a determination of the presence or amount of the analyte in the liquid sample, and optionally include reference binding elements;
   wherein at least a portion of the dissolvable elements is located closer to the center of the microarray and/or at least a portion of the dissolvable elements is located closer to the periphery of the microarray than each of the immobilized binding elements.

2. The device according to Aspect 1 wherein the assay elements comprise dissolvable target detection reagent and optionally, dissolvable reference reagent, all provided as at least a portion of the dissolvable elements.

3. The device according to Aspect 1 wherein the one or more analyte-related binding elements comprise respective analyte analogues, to which dissolvable target detection reagent is capable of binding.

4. The device according to any of Aspects 1 to 3, wherein the immobilized binding elements include reference binding elements and the dissolvable elements include dissolvable reference reagents adapted to bind to the reference binding elements, and wherein each dissolvable reference reagent is located either closer to the center of the microarray or to the periphery of the microarray than the reference binding element to which it is adapted to bind, optionally wherein a portion of the dissolvable reference reagents is located closer to the center of the microarray and another portion of the dissolvable reference reagents is located closer to the periphery of the microarray than the reference binding element to which they are adapted to bind, and wherein upon applying the liquid sample on the surface the dissolvable reference reagent bind to the reference binding element to which said reagent is adapted and produce a detectable reference signal.

5. The device as claimed in Aspect 4 wherein the reference binding elements are located in the microarray relative to the analyte related binding elements to provide the detectable reference signal that is subject to the same device and measurement conditions variations as the detectable analyte related signal.

6. The device as claimed in any of Aspects 1 to 5 wherein the detectable signal is optically detectable signal and the binding elements are located in the microarray on the detection surface to avoid or reduce optical distortion, preferably wherein the reference binding elements are located in the microarray on the detection surface to take into account the optical properties of one or more of the optical detection device and the detection surface thereby improving one or both of a standardization and a calibration signal.

7. The device according to any of Aspects 2 to 5, wherein each analyte-related binding element for an analyte is capable of binding the respective analyte and analyte analogue and each dissolvable target detection reagent comprises an analyte analogue and is capable of binding to the analyte-related binding element and also comprises a label capable for providing the analyte-related signal, and wherein each dissolvable target detection reagent competes with the analyte in the liquid sample for binding to the analyte-related binding element.

8. The device according to any of Aspects 2 to 5, wherein each analyte-related binding element is capable of binding the analyte and is also capable of binding the analyte analogue; the dissolvable target detection reagents comprise an analyte analogue that is conjugated to a molecule that can specifically bind to a binding agent to provide a labelled target compound and the device, preferably the microarray also comprises a labelled binding compound which comprises the binding agent and a label molecule for providing the analyte-related signal and is capable of binding the dissolvable target detection reagent, and wherein upon binding of the dissolvable target detection reagent to the analyte-related binding element the labelled binding compound also binds to the dissolvable target detection reagent, wherein the labelled target compound competes with the analyte for binding to the analyte-related binding element for the same analyte.

9. The device according to any of Aspects 2 to 5 wherein both the analyte-related binding elements and the dissolvable target detection reagents bind simultaneously and specifically to the same analytes at different binding sites, or to different ligands.

10. The device according to Aspect 9 wherein the dissolvable target detection reagents are labelled binding compounds and wherein upon binding of the analytes in the liquid sample to the analyte-related binding elements, the labelled binding compounds also bind to the bound analytes and thereby to the analyte-related binding elements.

11. The device according to any of Aspects 2 to 10 wherein a portion of the dissolvable target detection reagents is located closer to the center of the microarray and another portion of the dissolvable target detection reagents is located closer to the periphery of the microarray than the analyte-related binding element for the same analyte.

12. The device according to any of Aspects 1 to 10 wherein at least part of the dissolvable elements are located in the center of the microarray, so that preferably, when the liquid sample is applied to the center a later assay component located relatively closer to the periphery of the microarray is dissolved differently time-wise to early components that are located relatively closer to the centre of the microarray, and preferably dissolvable elements are co-located.

13. The device according to any of Aspects 5 to 10 wherein the periphery of the detection surface comprises only dissolvable elements so that it becomes free of said elements after the addition of the sample liquid on the detection surface so that there is a reduced risk that reflections from the side-walls would distort the optically detectable signal.

14. The device according to Aspects 1 wherein the dissolvable elements include functionalized magnetic particles.

15. A method for determining one or more of multiple analytes in a liquid sample using a device as claimed in any preceding claim, said method comprising the steps of:
applying a liquid sample to the detection surface to dissolve the dissolvable target detection reagents and the dissolvable reference reagents, when present, into the liquid sample, whereby at least a portion of the dissolvable target detection reagents binds to analyte and/or to analyte-related binding elements for the given analyte or at least a portion of the dissolvable target detection reagents binds to analyte and another portion to analyte related binding elements for the given analyte;
optionally removing the non-bound part of the liquid sample by washing;
detecting the analyte-related signal and optionally the reference signal; and
determining the presence or amount of analyte in the liquid sample from the detected analyte-related signal.

16. A method according to aspect 15, where the device includes a detection surface having thereon a microarray of elements comprising:
a plurality of dissolvable elements which are dissolvable upon applying a liquid sample to the detection surface, said dissolvable elements being immobilized by soluble binding at discrete locations in the microarray on the detection surface, wherein said dissolvable elements include assay elements comprising one or more dissolvable target detection reagents capable of binding to an analyte-related binding element or to the analyte or both and optionally dissolvable reference reagents;
a plurality of immobilized binding elements immobilized by insoluble binding at discrete locations in the microarray on the detection surface, wherein said immobilized binding elements include one or more of different analyte-related binding elements for each analyte and optionally include reference binding elements;
said analyte-related binding elements being adapted to bind one or both assay elements and analytes which, when bound, produce a detectable analyte-related signal dependent on the amount of the analyte in the liquid sample,
   wherein at least a portion of the dissolvable elements is located closer to the center of the microarray and/or a portion of the dissolvable elements is located closer to the periphery of the microarray than each of the immobilized binding elements, whereby once a liquid sample is applied on the detection surface the dissolvable elements are removed from the detection surface into the liquid, leaving a surface area covering the centre of the detection surface and/or a surface area proximal to the periphery of the detection surface, respectively, free of dissolvable elements, said method comprising the steps of:
applying a liquid sample to the detection surface to dissolve the dissolvable target detection reagents and the dissolvable reference reagents, when present, into the liquid sample, whereby at least a portion of the dissolvable target detection reagents binds to analyte and/or to analyte-related binding elements for the given analyte or at least a portion of the dissolvable target detection reagents binds to analyte and another portion to analyte related binding elements for the given analyte;
optionally removing the non-bound part of the liquid sample by washing;
detecting the analyte-related signal and optionally the reference signal; and
determining the presence or amount of analyte in the liquid sample from the detected analyte-related signal.

17. A method according to aspect 16, the method comprises washing to remove the non-bound part of the liquid sample.

18. A method according to aspect 16 or 17, wherein at least a portion of the dissolvable target detection reagents binds to analyte present in the liquid sample and/or at least a portion of the dissolvable target detection reagents binds to analyte analogues, said analyte analogues being bound to analyte related binding elements for the given analyte, wherein said analyte is either insolubly bound to the analyte related binding element or binds to the analyte related binding element upon applying the liquid sample.

19. A method according to any of aspects 16-18, wherein the dissolvable elements include dissolvable reference reagents adapted to bind to the reference binding elements as defined above, and wherein upon applying the liquid sample on the surface each dissolvable reference reagent binds to the reference binding element to which said element is adapted to bind, wherein said bound reference reagent results in a detectable reference signal, and said method also comprising: detecting the reference signal; determining a deviation of the detected reference signal from a predetermined reference signal; and adjusting the analyte-related signal in dependence on the determined deviation.

20. A method according to any of aspects 16-19, wherein the reference signal is subject to same device variations as the analyte-related signal.

21. A method according to any of aspects 16-20, wherein a device is used, wherein both the reference signal and the analyte-related signal are optically detectable signals and the immobilized binding elements are located on the detection surface so as to avoid or reduce optical distortion, preferably wherein the reference binding elements are located on the detection surface to take into account the optical properties of one or more of the optical detection device, the detection surface and the container, thereby improving one or both of a standardization and a normalization of the analyte-related signal.

22. A method according to any of aspects 16-21, wherein a device is used wherein each analyte-related binding element comprises an analyte analogue and thus the analyte-related binding element competes with the analyte for the dissolvable specific target detection reagent upon applying a liquid sample comprising the analyte, and wherein at least a portion of the dissolvable target detection reagents bind the analyte if present in the liquid sample, wherein optionally the bound analyte targets are removed by washing, and wherein dissolvable target detection reagents not bound to the analyte, binds to the target analyte-related binding elements, such that a higher concentration of analyte in the liquid sample results in a lower analyte-related signal level detectable from the respective analyte-related binding elements, and a lower concentration of analyte in the liquid sample results in a higher analyte-related signal level detectable from the same analyte-related binding elements.

23. A method according to any of aspects 16-22, wherein a device is used, wherein each analyte-related binding element for an analyte is capable of binding the respective analyte and analyte analogue and each dissolvable target detection reagent comprises an analyte analogue and is capable of binding to the analyte-related binding element and also comprises a label capable of providing a signal upon binding, wherein each dissolvable target detection reagent competes with the analyte in the liquid sample for binding to the analyte-related binding element, such that a higher concentration of analyte in the liquid sample results in a lower analyte-related signal level and a lower concentration of analyte in the liquid sample results in a higher analyte-related signal level, and determining therefrom the amount of analyte in the liquid sample based on a calibration generated from analyte-related signals obtained from a plurality of liquid samples containing different known amounts of analytes.

24. A method according to any of aspects 16-23, wherein a device is used wherein each analyte-related binding element is capable of binding the analyte and is also capable of binding the analyte analogue; the dissolvable target detection reagents comprise an analyte analogue that is conjugated to a molecule that can specifically bind to a binding agent that is conjugated to a labelling molecule to provide a labelled target compound and the device, preferably the microarray, also comprises a labelled binding compound which consists of the said binding agent and a label molecule for providing the analyte-related signal and is capable of binding a target detection reagent for which it is adapted to bind, and wherein upon binding of the target detection reagent to the analyte-related binding element the labelled binding compound also binds to the target detection reagent; wherein the labelled target compound competes with the analyte for binding to the analyte-related binding element for the same analyte such that a higher concentration of analyte in the liquid sample results in a lower analyte-related signal level and a lower concentration of analyte in the liquid results in a higher analyte-related signal level, and wherein preferably determining the amount of analytes in the liquid sample comprises determining the amount of the analyte related binding elements binding labelled target compounds, wherein from this and the total amount of analyte related binding elements on the assay surface the amount of analyte related binding elements binding analyte is determined from which the level of analyte in the liquid sample is assessed.

25. A method according to any of aspects 16-24, wherein a device is used wherein both the analyte-related binding elements and the dissolvable target detection reagents bind simultaneously and specifically to the same analytes at different binding sites, or to different ligands, preferably wherein the dissolvable target detection reagents are labelled binding compounds and wherein upon binding of the labelled binding compounds to the analytes that are bound to the analyte-related binding elements to which they are adapted to bind that the signal that evolves is related to the said analyte concentration, such that a higher concentration of analyte results in a higher analyte-related signal level and a lower concentration of analyte results in a lower analyte-related signal level and wherein preferably assessing the level of analytes in the liquid sample comprises determining the amount of labelled binding compounds bound to the analytes and thereby to the analyte related binding elements and from this the level of analyte in the liquid sample is assessed.

26. A method according to any of aspects 16-25, wherein a device as defined herein, in particular the device as described hereinafter, is used, being a device wherein a portion of the dissolvable target detection reagents is located closer to the center of the microarray and another portion of the dissolvable target detection reagents is located closer to the periphery of the microarray than the analyte-related binding element for the same analyte, a device, wherein a part of the dissolvable elements are located in an area of the detection surface comprising the center of the microarray and a part of the dissolvable elements are located in an area of the microarray located relatively closer to its periphery, so that preferably, when the liquid sample is applied to the center a “later” assay component located relatively closer to the periphery of the microarray is dissolved differently time-wise to the “early” components that are located relatively closer to the centre of the microarray, preferably dissolvable elements are co-located, which provides a more even distribution of these dissolvable elements, wherein preferably the liquid sample is applied to the center of the detection surface.

27. A method according to any of aspects 16-26, wherein a device is used, wherein the periphery of the microarray is proximal a container side-wall and comprises only dissolvable elements so that it becomes free of said elements after the addition of the sample liquid on the detection surface so that there is a reduced risk that reflections from the side-walls would distort an optical reading, wherein preferably the liquid sample is applied to the detection surface so that it is evenly distributed thereon.

28. A method according to any of aspects 16-27, wherein each of the one or more dissolvable target detection reagents is capable of binding an analyte, each being adapted to bind a different analyte.

29. A method according to any of aspects 16-28, wherein the total volume of the liquid sample to be measured is added to the detection surface, e.g. wells, in steps consisting of small volumes thus controlling certain parameters that influence conditions for the reactions inside the wells, supporting the optimization of the reactions.

30. A method according to any of aspects 16-29, wherein bound elements are detected by an optical detection device, and the reference binding elements, when present, are located in the microarray to take into account the optical properties of the optical detection device thereby avoiding or reducing errors in a standardization and calibration signal.

31. A device for use in the determination of one or more of multiple analytes in a liquid sample, said device including a detection surface for exposure to the liquid sample and having thereon a number of elements comprising:
a plurality of dissolvable elements which are dissolvable upon applying the liquid sample to the detection surface, said dissolvable elements being attached to the detection surface, such as by soluble binding, at first discrete locations on the detection surface, the dissolvable elements comprising one or more first components, and
a plurality of immobilized binding elements immobilized, such as by insoluble binding, at second discrete locations on the detection surface, each immobilized binding element comprises one or more binding components each being configured to attach to one or more of the multiple analytes and/or one or more of the first components;
   wherein at least a portion of the first discrete locations is located closer to a centre of the detection surface than each of the second discrete locations.

32. A device according to aspect 31, wherein all first discrete locations are closer to the centre than any of the second discrete locations.

33. A device according to aspect 31, wherein:
the dissolvable elements comprise a first group of dissolvable elements and a second group of dissolvable elements,
the first discrete locations of all dissolvable elements of the first group are positioned, on the detection surface, closer to the centre than the second discrete locations of any of the immobilized binding elements and
the second discrete positions of all of the immobilized binding elements are positioned, on the detection surface, closer to the centre than the first discrete positions of any of the dissolvable elements of the second group.

34. A device according to aspect 33, further comprising additional immobilized elements each positioned at a third discrete location, wherein the first discrete locations of all dissolvable elements of the second group are positioned, on the detection surface, closer to the centre than any of the third discrete locations.

35. A device according to any of aspects 31-34, wherein each second discrete location is positioned so that a first straight line through the centre and the pertaining second discrete location has an angle of no more than 10 degrees to a second straight line through the centre and a first discrete location.

36. A device according to any of aspects 31-35, further comprising, on the detection surface, a plurality of dissolvable reference elements which are dissolvable upon applying the liquid sample to the detection surface, the dissolvable reference elements comprising one or more reference components.

37. A device according to any of aspects 31-36, further comprising, on the detection surface, a plurality of immobilized reference elements at fifth discrete locations on the detection surface, each immobilized reference element comprising one or more reference binding components each being configured to attach to one or more of the multiple analytes and/or one or more of the reference components.

38. A device for use in the determination of one or more of multiple analytes in a liquid sample, said device including a detection surface for exposure to the liquid sample and having thereon a number of elements comprising:
a plurality of dissolvable elements which are dissolvable upon applying the liquid sample to the detection surface, said dissolvable elements being attached to the detection surface at first discrete locations on the detection surface, the dissolvable elements comprising one or more first components, and
a plurality of immobilized elements immobilized at second discrete locations on the detection surface, each immobilized element comprising one or more binding components each being configured to attach to one or more of the multiple analytes and/or one or more of the first components,
   wherein the detection surface has a centre, where a plurality of non-overlapping first areas exist on the detection surface, each first area being delimited at least partly by two straight lines through the centre, where each first area has the same amount of dissolvable elements and immobilized elements.

39. A device according to aspect 38, wherein the two straight lines, for at least two of the non-overlapping first areas, define at least substantially the same angle between them.

40. A device according to any of aspects 38-39, wherein at least two of the non-overlapping first areas have at least substantially the same areas.

41. A device according to any of aspects 38-40, wherein at least two of the non-overlapping first areas have the same number of first discrete locations.

42. A device according to aspect 41, wherein the first discrete locations in the at least two non-overlapping first areas define sets of first discrete locations, each set having one first discrete location in each of the at least two non-overlapping first areas, wherein the first discrete locations of each set are at least substantially at the same distance to the centre.

43. A device according to any of aspects 38-42, wherein at least two of the non-overlapping first areas have the same number of second discrete locations.

44. A device according to aspect 43, wherein the second discrete locations in the at least two non-overlapping first areas define sets of second discrete locations, each set having one second discrete location in each of the at least two non-overlapping first areas, wherein the second discrete locations of each set are at least substantially at the same distance to the centre.

45. A device according to any of aspects 38-44, wherein the number of first and second discrete locations are identical in the at least two non-overlapping first areas.

46. A device according to any of aspects 38-45, wherein two of the at least two non-overlapping first areas are positioned on opposite sides of the centre.

47. A device according to aspect 46, wherein the first and second discrete locations of the two non-overlapping first areas are mirrored around the centre.

48. A device according to any of aspects 38-47, wherein, in at least one of the non-overlapping first areas, all first discrete locations are closer to the centre than any of the second discrete locations.

49. A device according to aspect 48, wherein, in the at least one of the non-overlapping first areas:
the dissolvable elements comprise a first group of dissolvable elements and a second group of dissolvable elements,
the first discrete locations of all dissolvable elements of the first group are positioned, on the detection surface, closer to the centre than the second discrete locations of any of the immobilized binding elements and
the second discrete positions of all of the immobilized binding elements are positioned, on the detection surface, closer to the centre than the first discrete positions of any of the dissolvable elements of the second group.

50. A device according to aspect 49, further comprising, in the at least one of the non-overlapping first areas, additional immobilized elements each positioned at a third discrete location, wherein the first discrete locations of all dissolvable elements of the second group are positioned, on the detection surface, closer to the centre than any of the third discrete locations.

51. A device according to any of aspects 38-50, comprising two second, non-overlapping areas, which are non-overlapping also with the first non-overlapping areas, where the first and second non-overlapping areas have:
different numbers of first discrete locations,
different numbers of second discrete locations,
different distances from the first discrete locations to the centre,
different distances from the second discrete locations to the centre,
different areas,
different angles between the two straight lines at least partly delimiting the pertaining area,
different first components of the dissolvable elements, and/or
different binding components of the immobilized elements

52. A device according to aspect 51, wherein the number of first and second discrete locations are identical in the two non-overlapping second areas.

53. A device according to any of aspects 51 and 52, wherein the two non-overlapping second areas are positioned on opposite sides of the centre.

54. A device according to aspect 53, wherein the first and second discrete locations of the two non-overlapping second areas are mirrored around the centre.

55. A device according to any of aspects 31-54, further comprising elements configured to guide the liquid sample along at least substantially straight paths from the centre and radially therefrom.

56. A device according to any of aspects 55, wherein the detection surface is plane.

57. A device according to any of aspects 31-56, wherein the detection surface is cone-shaped.

58. A device according to any of aspects 31-57, further comprising, on the detection surface, a plurality of dissolvable reference elements which are dissolvable upon applying the liquid sample to the detection surface, the dissolvable reference elements comprising one or more reference components.

59. A device according to any of aspects 31-58, further comprising, on the detection surface, a plurality of immobilized reference elements, each immobilized reference element comprising one or more reference binding components each being configured to attach to one or more of the multiple analytes and/or one or more of the reference components.

60. A device according to any of aspects 31-59, wherein the binding components include one or more of different analyte-related binding components for each analyte, any or each of said analyte-related binding components being adapted to attach to a first component and/or an analyte.

61. The device according to aspect 60, wherein the one or more analyte-related binding components comprise respective analyte analogues, to which the one or more first components is capable of attaching.

62. The device according to any of aspects 31-61, wherein each binding component is configured to attach to an analyte or an analyte analogue and a first component, and further comprises a label capable of generating an output signal.

63. The device according to any of aspects 31-62, wherein each binding component is capable of attaching to:
an analyte,
an analyte analogue and
one of the one or more first components, comprising an analyte analogue that is conjugated to a molecule that can specifically attach to the pertaining binding component.

64. The device according to any of aspects 31-63, wherein both the binding components and the first components attach simultaneously and specifically to the same analytes at different binding sites, or to different ligands.

65. The device according to aspect 64, wherein the first components are labelled binding compounds and wherein, upon binding of the analytes in the liquid sample to the binding components, the labelled binding compounds also attach to the attached analytes and thereby to the binding components.

66. The device according to any of aspects 31-65, wherein a portion of the first components is located closer to the centre and another portion of the first components is located closer to a periphery of the detection surface than the binding component for the same analyte.

67. The device according to any of aspects 31-66, wherein at least part of the dissolvable elements are located in the centre of the detection surface, so that preferably, when the liquid sample is applied to the centre a later assay component located relatively closer to the periphery of the microarray is dissolved differently time-wise to early components that are located relatively closer to the centre of the detection surface, and preferably dissolvable elements are co-located.

68. The device according to any of aspects 31-67, wherein a periphery of the detection surface comprises only dissolvable elements so that it becomes free of said elements after the addition of the sample liquid on the detection surface so that there is a reduced risk that reflections from the side-walls would distort the optically detectable signal.

69. The device according to any of aspects 31-68, wherein the dissolvable elements include functionalized magnetic particles.

70. A method for determining one or more of multiple analytes in a liquid sample using a device according to any of the preceding aspects, said method comprising the steps of:
a) applying a liquid sample to or at the centre of the detection surface,
b) guiding the liquid sample to the dissolvable elements to at least partly dissolve the dissolvable elements to release the one or more first components into the liquid sample,
c) guiding the liquid sample with the one or more first components to the immobilized elements to have the one or more binding components attach to the one or more of the multiple analytes and/or the one or more of the first components,
d) detecting a signal output by
   the one or more binding components attached to the one or more of the multiple analytes and/or the one or more of the first components and/or
   the one or more of the multiple analytes and/or the one or more of the first components attached to the one or more binding components; and
e) determining, based on the signal, the presence or amount of at least one of the multiple analytes in the liquid sample.

71. A method according to aspect 70, wherein at least one of the guiding steps comprises rotating the disc around an axis provided through the centre.

72. A method according to any of aspects 70 and 71, further comprising, during at least one of the guiding steps, vibrating the detection surface.

73. A method according to aspect 71, wherein at least a portion of the first discrete locations is located closer to a centre of the detection surface than each of the second discrete locations and wherein the guiding steps comprise guiding the liquid sample in a direction away from the centre.

74. A method according to any of aspects 70-73, wherein the detection surface has a centre, where a plurality of non-overlapping first areas exist on the detection surface, each first area being delimited at least partly by two straight lines through the centre, where each first area has the same amount of dissolvable elements and immobilized elements and wherein the guiding steps comprise guiding the liquid sample at least substantially simultaneously over the at least two non-overlapping areas.

75. A method according to aspect 74, wherein the guiding steps comprise essentially guiding different portions of the liquid sample inside respective first non-overlapping areas.

76. A method according to any of aspects 70-75, wherein the applying step comprises maintaining the applying of liquid sample until liquid sample has reached all first and second discrete locations.

77. A method according to any of aspects 70-76, further comprising the step of, between the guiding steps and the detection step, flowing a second liquid over the detection surface and the first and second discrete locations.

78. A method according to any of aspects 70-77, wherein the guiding step b) comprises attaching an analyte of the liquid sample to a first component of a dissolvable element.

79. A method according to aspect 78, wherein guiding step c) comprises attaching the analyte or the first component to a binding component of an immobilized element.

80. A method according to any of aspects 70-79, wherein the device further comprises, on the detection surface, a plurality of dissolvable reference elements which are dissolvable upon applying the liquid sample to the detection surface, the dissolvable reference elements comprising one or more reference components and wherein the detecting step comprises detecting a second signal output by the one or more reference components.

81. A method according to any of aspects 70-80, wherein the device further comprises, on the detection surface:
a plurality of dissolvable reference elements which are dissolvable upon applying the liquid sample to the detection surface, the dissolvable reference elements comprising one or more reference components and
a plurality of immobilized reference elements and having one or more reference binding components,
   the method comprising the additional guiding step of guiding liquid sample from the dissolvable reference elements to the immobilized reference elements to the one or more reference binding components attach to one or more of the multiple analytes and/or one or more of the reference components, and
   wherein the detecting step comprises detecting a second signal output by the binding components and/or the one or more analytes and/or reference components attached to the binding components.

82. A method according to any of aspects 70-81, wherein the detecting step comprises launching first radiation toward the detecting surface and detecting second radiation received from:
the one or more binding components attached to the one or more of the multiple analytes and/or the one or more of the first components
the one or more of the multiple analytes and/or the one or more of the first components attached to the one or more binding components and/or
a marker attached to the one or more binding components, the one or more of the multiple analytes and/or the one or more first components.

83. A method according to aspect 82, wherein the second radiation is generated by:
the one or more binding components attached to the one or more of the multiple analytes and/or the one or more of the first components and/or
the one or more of the multiple analytes and/or the one or more of the first components attached to the one or more binding components absorbing a portion of the first radiation and transmitting/reflecting/scattering/outputting, as the second radiation, another portion of the first radiation.

84. A method according to aspect 82, wherein the second radiation is generated by:
the one or more binding components attached to the one or more of the multiple analytes and/or the one or more of the first components and/or
the one or more of the multiple analytes and/or the one or more of the first components attached to the one or more binding components,
   absorbing a portion of the first absorbing a portion of the first radiation and emitting, as the second radiation, fluorescence.

1. A device for use in determination of one or more of multiple analytes in a liquid sample, the device comprising:
a plane detection surface for exposure to the liquid sample and having thereon a plurality of elements, the plurality of elements including
a plurality of dissolvable elements which are dissolvable upon applying the liquid sample to the plane detection surface, the plurality of dissolvable elements being attached to the plane detection surface at first discrete locations on the plane detection surface, the plurality of dissolvable elements including one or more first components, and
a plurality of immobilized binding elements immobilized at second discrete locations on the plane detection surface, each immobilized binding element including one or more binding components, each binding component configured to attach to one or more analytes of the multiple analytes and/or the one or more first components,
wherein at least a portion of the first discrete locations is located closer to a centre of the plane detection surface than each of the second discrete locations.

2. The device according to claim 1, wherein all of the first discrete locations are closer to the centre than any of the second discrete locations.

3. The device according to claim 1, wherein each second discrete location is positioned so that a first straight line through the centre and the second discrete location has an angle of no more than 10 degrees to a second straight line through the centre and at least one first discrete location.

4. A device for use in determination of one or more of multiple analytes in a liquid sample, the device comprising:
a plane detection surface for exposure to the liquid sample and having thereon a plurality of elements, a plurality of elements including
a plurality of dissolvable elements which are dissolvable upon applying the liquid sample to the plane detection surface, said dissolvable elements being attached to the plane detection surface, by soluble binding, at first discrete locations on the plane detection surface, the plurality of dissolvable elements including one or more first components, and
a plurality of immobilized elements immobilized at second discrete locations on the plane detection surface, each immobilized element including one or more binding components, each binding component configured to attach to one or more analytes of the multiple analytes and/or the one or more first components,
wherein the plane detection surface has a centre, where a plurality of non-overlapping first areas exist on the plane detection surface, each first area being delimited at least partly by two straight lines through the centre, where each first area has a same amount of dissolvable elements and immobilized elements.

5. The device according to claim 4, wherein at least two first area of the plurality of non-overlapping first areas each have at least substantially a same area.

6. The device according to claim 4, wherein at least two first areas of the plurality of non-overlapping first areas each have a same amount of first discrete locations.

7. The device according to claim 4, wherein at least two first area of the plurality of non-overlapping first areas each have a same amount of second discrete locations.

8. The device according to claim 4, comprising two non-overlapping second areas, the two non-overlapping second areas are non-overlapping also with the plurality of non-overlapping first areas, where the plurality of non-overlapping first areas and the two non-overlapping second areas have:
different numbers of respective first discrete locations,
different numbers of respective second discrete locations,
different distances from the respective first discrete locations to the centre,
different distances from the respective second discrete locations to the centre,
different respective areas,
different angles between respective two straight lines at least partly delimiting the respective areas,
different first components of the plurality of dissolvable elements, and/or
different binding components of the plurality of immobilized elements.

9. The device according to claim 4, wherein each binding component is configured to attach to:
an analyte,
an analyte analogue, and
one of the one or more first components, comprising an analyte analogue that is conjugated to a molecule that can specifically attach to the binding component.

10. A method for determining one or more analytes of multiple analytes in a liquid sample using the device according to claim 1, the method comprising:
a) applying the liquid sample to or at the centre of the plane detection surface,
b) performing a first guiding step of guiding the liquid sample to the plurality of dissolvable elements to at least partly dissolve the plurality of dissolvable elements to release the one or more first components into the liquid sample,
c) performing a second guiding step of guiding the liquid sample with the one or more first components to the plurality of immobilized biding elements to have the one or more binding components attach to the one or more analytes of the multiple analytes and/or the one or more first components,
d) detecting a signal, the signal output by
the one or more binding components attached to the one or more of the multiple analytes and/or the one or more first components, and/or
the one or more analytes and/or the one or more first components attached to the one or more binding components; and
e) determining, based on the signal, a presence or amount of at least one analyte of the multiple analytes in the liquid sample.

11. The method according to claim 10, wherein
the first guiding step and the second guiding step each include guiding the liquid sample in a direction away from the centre.

12. The method according to claim 10, wherein
a plurality of non-overlapping first areas exist on the plane detection surface, each first area being delimited at least partly by two straight lines through the centre, where each first area has a same amount of dissolvable elements and immobilized elements, and
the first guiding step and the second guiding step each include guiding the liquid sample at least substantially simultaneously over the plurality of non-overlapping first areas.

13. The method according to claim 10, wherein
the device further comprises, on the plane detection surface:
a plurality of dissolvable reference elements which are dissolvable upon applying the liquid sample to the plane detection surface, the plurality of dissolvable reference elements comprising one or more reference components, and
a plurality of immobilized reference elements and having one or more reference binding components,
the method includes an additional guiding step of guiding the liquid sample from the plurality of dissolvable reference elements to the plurality of immobilized reference elements to the one or more reference binding components, the one or more reference binding components each configured to attach to at least one analyte of the multiple analytes and/or the one or more reference components, and
the detecting the signal comprises detecting a second signal output by the one or more binding components and/or the one or more analytes and/or the one or more reference components attached to the one or more binding components.

14. The method according to claim 10, wherein the detecting the signal comprises launching first radiation toward the plane detection surface and detecting second radiation received from:
the one or more binding components attached to the one or more analytes and/or the one or more first components,
the one or more analytes and/or the one or more first components attached to the one or more binding components, and/or
a marker attached to the one or more binding components, the one or more analytes and/or the one or more first components.

15. A measurement system for simultaneous determination of one or more analytes, the measurement system comprising:
the device according to according to claim 1;
a pipette configured to dispense a liquid sample at the center of the plane detection surface of the device,
an illumination light source configured to emit light toward the device to induce one or more analyte related signals from the device as light signals;
an optical detection device configured to detect an intensity and location of the light signals; and
an analysis unit configured to analyze the detected intensity and location of the light signals to determine a presence or amount of the one or more analytes in the liquid sample.
